(19) United States
(12) Patent Application Publication
(10) Pub. No.: US 2024/0274301 A1
(43) Pub. Date: Aug. 15, 2024

(54) SYSTEMS AND METHODS FOR CLINICAL CLUSTER IDENTIFICATION INCORPORATING EXTERNAL VARIABLES

(71) Applicant: nference, Inc., Cambridge, MA (US)

(72) Inventors: Samir Awasthi, Jamaica Plain, MA (US); John Rincon-Hekking, Cambridge, MA (US)

(73) Assignee: nference, Inc., Cambridge, MA (US)

(21) Appl. No.: 18/437,795

(22) Filed: Feb. 9, 2024

Related U.S. Application Data
(60) Provisional application No. 63/484,376, filed on Feb. 10, 2023.

(51) Int. Cl. G16H 50/70

(52) U.S. Cl. CPC G16H 50/70

(57) ABSTRACT

An apparatus for identifying clusters based on augmented data sets, the apparatus including at least a processor and a memory communicatively connected to the at least a processor, the memory containing instructions configuring the at least a processor to receive one or more vitality data sets, retrieve auxiliary information for the one or more vitality data sets, generate one or more augmented data sets as a function of the auxiliary information and the one or more vitality data sets, identify at least one cluster based on the one or more augmented data sets and provide the at least one cluster to a cluster analysis platform, wherein the cluster analysis platform is configured to generate a similarity datum as a function of the at least one cluster and the one or more augmented data sets.

### CROSS-REFERENCE TO RELATED APPLICATIONS

This application claims the benefit of priority of U.S. Provisional Patent Application Ser. No. 63/484,376, filed on Feb. 10, 2023, and titled “SYSTEMS AND METHODS FOR CLINICAL COHORT IDENTIFICATION INCORPORATING EXTERNAL VARIABLES” which is incorporated by reference herein in its entirety.

### FIELD OF THE INVENTION

This application relates generally to identifying clinical clusters based on vitality data sets and specifically to techniques for identifying clinical clusters that incorporate variables from external sources other than the sources of the vitality data sets.

### BACKGROUND

Current systems used to identify clinical clusters are lacking and cannot accommodate missing variables. As a result, outputs of these systems may be inaccurate and/or limited to the inputs that were received.

### SUMMARY OF THE DISCLOSURE

In an aspect an apparatus for identifying clusters based on augmented data sets is described. The apparatus includes at least a processor and a memory communicatively connected to the at least a processor. The memory contains instructions configuring the at least a processor to receive one or more vitality data sets, retrieve auxiliary information for the one or more vitality data sets, generate one or more augmented data sets as a function of the auxiliary information and the one or more vitality data sets, identify at least one cluster based on the one or more augmented data sets and provide the at least one cluster to a cluster analysis platform, wherein the cluster analysis platform is configured to generate a similarity datum as a function of the at least one cluster and the one or more augmented data sets.

In another aspect, a method for identifying clusters based on augmented data sets described. The method includes receiving by at least a processor, one or more vitality data sets, retrieving, by at least a processor, auxiliary information for the one or more vitality data sets, generating, by at least a processor, one or more augmented data sets as a function of the auxiliary information and the one or more vitality data sets, identifying, by at least a processor, at least one cluster based on the one or more augmented data sets and providing, by at least a processor, the at least one cluster to a cluster analysis platform, wherein the cluster analysis platform is configured to generate a similarity datum as a function of the at least one cluster and the one or more augmented data sets.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is an exemplary embodiment of an apparatus for identifying clusters based on augmented health records;

FIG. 2 is a simplified diagram of a system for identifying clinical clusters according to some embodiments;

FIG. 3 is a simplified diagram of a data flow for identifying clinical clusters according to some embodiments.

FIG. 4 is a simplified diagram of a method for identifying clinical clusters according to some embodiments;

FIG. 5 is a simplified diagram of a method for generating cost of care data and merging cost of care data with electronic health records according to some embodiments;

FIGS. 6-10 are simplified diagrams of methods for generating cost of care data and merging cost of care data with electronic health records according to some embodiments;

FIG. 11 is a block diagram of an exemplary machine-learning process;

FIG. 12 is a diagram of an exemplary embodiment of a neural network;

FIG. 13 is a diagram of an exemplary embodiment of a node of a neural network;

FIG. 14 is an exemplary embodiment of a method for identifying clusters based on augmented health records; and

FIG. 15 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Health records, such as electronic health records (EHRs) and patient charts, provide a vast trove of data that can be exploited for a variety of biomedical applications. However, health records are often represented in an unstructured or semi-structured format, such as the text of a note written by a physician. As a result, it can be challenging to use automated techniques to organize information from health records in a useful way.

Often, it is desirable to identify one or more clinical clusters in a set of health records. For example, a clinical cluster may include a set of patients that have one or more relevant clinical traits in common, such as patients in a certain age range that have received a common diagnosis. Once identified, a clinical cluster can be used to conduct studies, to train neural network models, or for a variety of other biomedical applications.

Curation of health records to identify clinical clusters can be performed in several ways, including curation by domain experts, using heuristic algorithms, or a combination thereof (e.g., applying a heuristic filtering algorithm followed by expert validation). However, identifying clinical clusters using these techniques may present certain limitations. For example, in some applications, it is desirable to identify clinical clusters based on information that is not typically found in patient health records.

Accordingly, it is desirable to develop improved techniques for identifying clinical clusters that can address gaps in information found in patient health records.

Over the last several years, an increasing amount of clinical patient data has become digitized, and progress has been made in building systems that process clinical patient data in an electronic format. For example, cloud-based systems can collect digitized health records from across institutions (e.g., hospitals, clinics, etc.) and can analyze patient data at a scale that was not attainable until recently. For example, de-identification of electronic health records has opened up the possibility of learning from de-identified data within and across medical institutions/facilities that are owners of the de-identified data. Variations of federated learning approaches are now being used to facilitate such learning within and across data repositories.

As the amount of data and processing scale increases, there is an emerging need for techniques that can automate the process of organizing and curating health records in a computationally efficient manner. One exemplary use of curated health records is for identifying clusters, e.g., clinical patient data associated with patients that share one or more desired attributes. For example, a first cluster may include patients that have been diagnosed with a particular disease, and another cluster may include a control group of patients that were not diagnosed with that disease. These clusters can be analyzed and used for a variety of applications. For example, they can be used as labeled training data to train a neural network model to predict whether a given patient is likely to be diagnosed with the disease. In this example, the input to the neural network model may the health records of the patients each of the clusters, or information extracted from the health records, and the labels used for training may include the identification of which cluster each patient belongs to (e.g., whether a patient was or was not diagnosed with the disease).

The types of information found in electronic health records can vary among patients and institutions, and may illustratively include patient data (e.g., age, gender, etc.), medical test results, disease diagnoses, prescribed medications, and the like. The electronic health records may be obtained from a single source (e.g., a single medical institution) or may be aggregated across disparate sources and may span multiple media types. Nevertheless, the information found in electronic health records may provide an incomplete picture of the care given to a patient. Such gaps in information may limit the types of analysis that can be performed using electronic health records, which may in turn limit the efficacy of downstream tasks such as neural network model training. For example, electronic health records may not identify the costs associated with the care delivered to a patient. Indeed, the patient's cost of care may be determined by a different institution (e.g., an insurance company), such that the source of cost information may be administratively and physically distinct from the source of the patient's electronic health records.

Therefore, an opportunity exists for learning within and across data repositories of deidentified data by leveraging sources external to these de-identified data repositories. The external sources may fill gaps in the electronic health records, such as missing cost information. In turn, this may enable more complete and efficient performance of downstream tasks, such as cluster identification.

However, there are several challenges to be overcome to incorporate external variables into a process of identifying clinical clusters based on electronic health records, particularly when the external variables are at least in part unstructured, span media types, and/or are made up of multiple variables. Accordingly, it is desirable to develop improved techniques for identifying clinical clusters that can efficiently incorporate external variables.

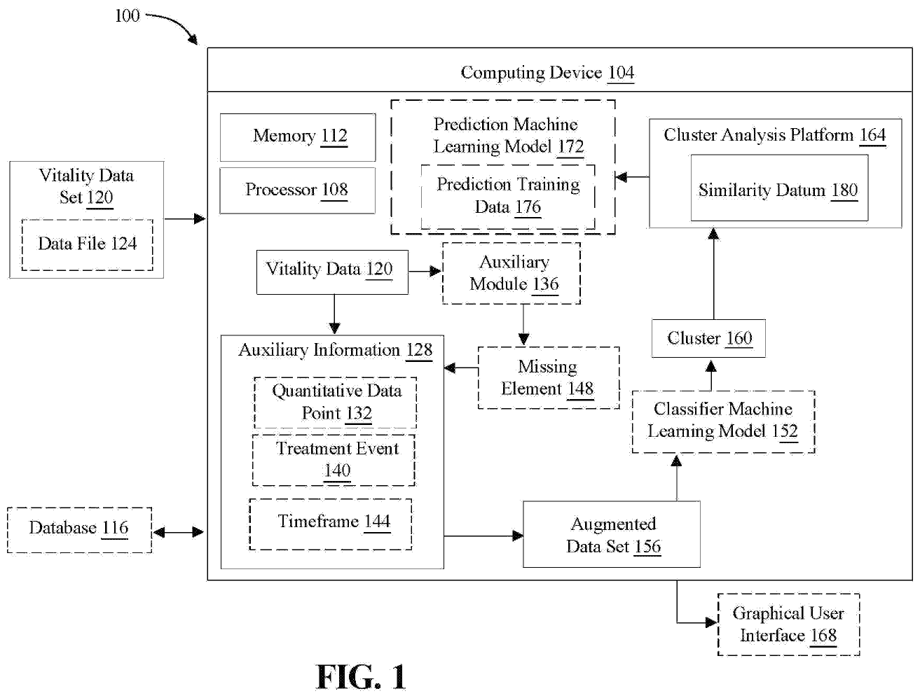

Referring now to FIG. 1, apparatus 100 for identifying a cluster based on augmented health records is described. Apparatus 100 includes a computing device 104. Apparatus 100 includes a processor 108. Processor 108 may include, without limitation, any processor 108 described in this disclosure. Processor 108 may be included in a and/or consistent with computing device 104. In one or more embodiments, processor 108 may include a multi-core processor. In one or more embodiments, multi-core processor may include multiple processor cores and/or individual processing units. “Processing unit” for the purposes of this disclosure is a device that is capable of executing instructions and performing calculations for a computing device 104. In one or more embodiments, processing units may retrieve instructions from a memory, decode the data, secure functions and transmit the functions back to the memory. In one or more embodiments, processing units may include an arithmetic logic unit (ALU) wherein the ALU is responsible for carrying out arithmetic and logical operations. This may include, addition, subtraction, multiplication, comparing two data, contrasting two data and the like. In one or more embodiments, the processing unit may include a control unit wherein the control unit manages execution of instructions such that they are performed in the correct order. In none or more embodiments, the processing unit may include registers wherein the registers may be used for temporary storage of data such as inputs fed into the processor and/or outputs executed by the processor. In one or more embodiments, the processing unit may include cache memory wherein memory may be retrieved from cache memory for retrieval of data. In one or more embodiments, the processing unit may include a clock register wherein the clock register is configured to synchronize the processor with other computing components. In one or more embodiments, processor 108 may include more than one processing units having at least one or more arithmetic and logic units (ALUs) with hardware components that may perform arithmetic and logic operations. Processing units may further include registers to hold operands and results, as well as potentially “reservation station” queues of registers, registers to store interim results in multi-cycle operations, and an instruction unit/control circuit (including e.g. a finite state machine and/or multiplexor) that reads op codes from program instruction register banks and/or receives those op codes and enables registers/arithmetic and logic operators to read/output values. In one or more embodiments, the processing unit may include a floating-point unit (FPU) wherein the FPU is configured to handle arithmetic operations with floating point numbers. In one or more embodiments, processor 108 may include a plurality of processing units wherein each processing unit may be configured for a particular task and/or function. In one or more embodiments, each core within multi-core processor may function independently. In one or more embodiments, each core within multi-core processor may perform functions in parallel with other cores. In one or more embodiments, multi-core processor may allow for a dedicated core for each program and/or software running on a computing system. In one or more embodiments, multiple cores may be used for a singular function and/or multiple functions. In one or more embodiments, multi-core processor may allow for a computing system to perform differing functions in parallel. In one or more embodiments, processor 108 may include a plurality of multi-core processors. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing devices operating in concert, in parallel, sequentially or the like; two or more computing devices may be included together in a single computing device 104 or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices in a first location and a second computing device 104 or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device 104, which may operate in parallel, in series, redundantly, or in any other manner used for distribution of tasks or memory 112 between computing devices. Computing device 104 may be implemented, as a non-limiting example, using a “shared nothing” architecture.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 may perform determinations, classification, and/or analysis steps, methods, processes, or the like as described in this disclosure using machine-learning processes. A “machine-learning process,” as used in this disclosure, is a process that automatedly uses a body of data known as “training data” and/or a “training set” (described further below in this disclosure) to generate an algorithm that will be performed by a Processor module to produce outputs given data provided as inputs; this is in contrast to a non-machine learning software program where the commands to be executed are determined in advance by a user and written in a programming language. A machine-learning process may utilize supervised, unsupervised, lazy-learning processes and/or neural networks, described further below.

With continued reference to FIG. 1, apparatus 100 includes a memory 112 communicatively connected to processor 108, wherein the memory 112 contains instructions configuring processor 108 to perform any processing steps as described herein. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device 104. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 112 may include a primary memory and a secondary memory. “Primary memory” also known as “random access memory” (RAM) for the purposes of this disclosure is a short-term storage device in which information is processed. In one or more embodiments, during use of computing device 104, instructions and/or information may be transmitted to primary memory wherein information may be processed. In one or more embodiments, information may only be populated within primary memory while a particular software is running. In one or more embodiments, information within primary memory is wiped and/or removed after computing device 104 has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 108 may access the information from primary memory.

Still referring to FIG. 1, Apparatus 100 may include database 116. Database may include a remote database 116. Database 116 may be implemented, without limitation, as a relational database, a key-value retrieval database such as a NOSQL database, or any other format or structure for use as database that a person skilled in the art would recognize as suitable upon review of the entirety of this disclosure. Database may alternatively or additionally be implemented using a distributed data storage protocol and/or data structure, such as a distributed hash table or the like. Database 116 may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, apparatus 100 may include and/or be communicatively connected to a server, such as but not limited to, a remote server, a cloud server, a network server and the like. In one or more embodiments, computing device 104 may be configured to transmit one or more processes to be executed by server. In one or more embodiments, the server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, the server may be configured to perform one or more processes as described below to allow for increased computational power and/or decreased power usage by system computing device 104. In one or more embodiments, computing device 104 may transmit processes to server wherein computing device 104 may conserve power or energy.

With continued reference to FIG. 1, apparatus 100 may include a host circuit. Host circuit includes at least a processor 108 communicatively connected to a memory 112. As used in this disclosure, a “host circuit” is an integrated circuit or a collection of interconnected circuits designed to manage, control, and/or interface with one or more functionalities in a system. In a non-limiting example, host circuit may be configured as a primary platform or base that provides essential infrastructure, resources, and interfaces to facilitate the operation of other connected or integrated components. Hosting circuit may include any computing device as described in this disclosure, including without limitation a microcontroller, microprocessor, digital signal processor (DSP) and/or system on a chip (SoC) that provide one or more services, resources, or data to other computing devices. Host circuit may include, be included in, and/or communicate with a mobile device such as a mobile telephone or smartphone. Host circuit may include a single computing device operating independently, or may include two or more computing device operating in concert, in parallel, sequentially or the like; two or more computing devices may be included together in a single computing device or in two or more computing devices. In some cases, Host circuit may include but is not limited to, for example, a computing device or cluster of computing devices in a first location and a second computing device or cluster of computing devices in a second location. In other cases, host circuit may include a main unit or a primary circuit in a network that controls communications and/or provide a central point of interface. In one or more embodiments, the host circuit may be used in lieu of computing device 104. In one or mor embodiments, the host circuit may carry out one or more processes as described in this disclosure intended for computing device 104.

With continued reference to FIG. 1, the processor is configured to receive vitality data sets 120. A “Vitality data set,” for the purposes of this disclosure is one or more digital files containing medical information associated with a patient. For example, and without limitation, vitality data set 120 may include information associated with a doctor's visit. In one or more embodiments, vitality data set 120 may include but is not limited to, information associated with a doctors visit, information associated with a procedure, information associated with blood test results, information associated with a hospital visit, information associated with prescriptions made to a patient and the like. In one or more embodiments, each vitality data set 120 may be associated with a patient. “Patient” for the purposes of this disclosure is an individual who has sought or is currently seeking medical treatment. For example, and without limitation, patients may include an individual who has undergone a medical procedure, an individual who has sought medical advice, an individual who has received medication and the like. In one or more embodiments, each vitality data set 120 may contain information describing an individual's medical history, clinical history and the like. In one or more embodiments, each vitality data set 120 may include but is not limited to, medications taken medications received, office visits, surgeries, diagnosis, diagnostic exams, lab work, medical treatments received, results as part of a blood test, urine test, DNA test and the like. In one or more embodiments, vitality data set 120 may include background information about a patient such as but not limited, a name, an address, a date of birth, a country of origin, race, ethnicity, religious observance, gender, sex, a country of residence, a state of residence, a city of residence, a social security number, and the like. In one or more embodiments, vitality data set 120 may include information about the particular medical insurance the patient currently has or had in the past. In one or more embodiments, vitality data set 120 may further include the current health status of the individual, such as but not limited to, current medical diagnosis, current medications being taken, upcoming medical visits, upcoming medical treatment, upcoming medical procedures, the fitness level of the patient (e.g., whether the patient is active) and the like. In one or more embodiments, vitality data set 120 may further include height, weight, skin tone, muscle mass, fat mass, body mass index (BMI) and the like. In one or more embodiments, vitality data set 120 may include any electronic health records as described in this disclosure.

With continued reference to FIG. 1, in one or more embodiments, vitality data set 120 may include notes written by medical professionals associated with the patients, such as notes regarding intake or the patient's current medical state. In one or more embodiments, vitality data set 120 may include handwritten notes that have been digitally scanned and are currently in a digital form. In one or more embodiments, vitality data set 120 may include images, X-rays, medical charts, notes written by a medical professional and the like.

With continued reference to FIG. 1, in one or more embodiments, vitality data set may be received by a patient, a medical professional and/or in any other way as described in this disclosure. In one or more embodiments, vitality data set may be received from database 116, wherein database 116 may include a database containing medical records of patients. In one or more embodiments, vitality data set may be received from one or more electronic health record systems wherein data may be integrated into vitality data sets 116. In one or more embodiments, processor 108 may be configured to receive information from one or more electronic heath record systems and store them in a database table wherein processor 108 may be configured to receive information from the database table to create vitality data set 116. In one or more embodiments, vitality data sets may be received by insurance providers, medical professionals, and the like. In one or more embodiments, medical records may be aggregated in database 116 from a plurality of locations and transmitted to computing device 104. In one or more embodiments, vitality data set may be received as a function of user input. “User input” for the purpose of this disclosure is an input of information into computing device by an individual. For example, and without limitation, user input may include the pressing of a key on a keyboard, movement on a mouse, transmission of digital information through the use of a web browser, portable hard drive and the like. A “User,” for the purposes of this disclosure is an individual who interacts with apparatus 100. For example, and without limitation, users may include a medical professional, a business associated with medical records, a lab technician, a research scientist and the like.

With continued reference to FIG. 1, processor 108 may be configured to retrieve a plurality of vitality data sets 120 wherein each vitality data set 120 may be associated with a patient. In one or more embodiments, vitality data set 120 may detail a patient's medical history, physical history and the like. In one or more embodiments, vitality data set 120 may include at least a data file 124. In one or more embodiments, data file 124 may include but is not limited to, an image, a document, a video and the like. In one or more embodiments, data file 124 may include handwritten and/or typed information. In one or more embodiments, processor 108 may be configured to extract textual data from data file 124 wherein textual data includes any information conveyed within a written or printed form. In one or more embodiments, processor 108 may be configured to extract textual data in order to retrieve information from within data file 124 for processing. In one or more embodiments, processor 108 may use optical character recognition to extract textual data from one or more vitality data sets 120.

Still referring to FIG. 1, in some embodiments, optical character recognition or optical character reader (OCR) includes automatic conversion of images of written (e.g., typed, handwritten or printed text) into machine-encoded text. In some cases, recognition of at least a keyword from an image component may include one or more processes, including without limitation optical character recognition (OCR), optical word recognition, intelligent character recognition, intelligent word recognition, and the like. In some cases, OCR may recognize written text, one glyph or character at a time. In some cases, optical word recognition may recognize written text, one word at a time, for example, for languages that use a space as a word divider. In some cases, intelligent character recognition (ICR) may recognize written text one glyph or character at a time, for instance by employing machine learning processes. In some cases, intelligent word recognition (IWR) may recognize written text, one word at a time, for instance by employing machine learning processes.

Still referring to FIG. 1, in some cases OCR may be an “offline” process, which analyses a static document or image frame. In some cases, handwriting movement analysis can be used as input to handwriting recognition. For example, instead of merely using shapes of glyphs and words, this technique may capture motions, such as the order in which segments are drawn, the direction, and the pattern of putting the pen down and lifting it. This additional information can make handwriting recognition more accurate. In some cases, this technology may be referred to as “online” character recognition, dynamic character recognition, real-time character recognition, and intelligent character recognition.

Still referring to FIG. 1, in some cases, OCR processes may employ pre-processing of image component. Pre-processing process may include without limitation de-skew, de-speckle, binarization, line removal, layout analysis or “zoning,” line and word detection, script recognition, character isolation or “segmentation,” and normalization. In some cases, a de-skew process may include applying a transform (e.g., homography or affine transform) to image component to align text. In some cases, a de-speckle process may include removing positive and negative spots and/or smoothing edges. In some cases, a binarization process may include converting an image from color or greyscale to black-and-white (i.e., a binary image). Binarization may be performed as a simple way of separating text (or any other desired image component) from a background of image component. In some cases, binarization may be required for example if an employed OCR algorithm only works on binary images. In some cases. a line removal process may include removal of non-glyph or non-character imagery (e.g., boxes and lines). In some cases, a layout analysis or “zoning” process may identify columns, paragraphs, captions, and the like as distinct blocks. In some cases, a line and word detection process may establish a baseline for word and character shapes and separate words, if necessary. In some cases, a script recognition process may, for example in multilingual documents, identify script allowing an appropriate OCR algorithm to be selected. In some cases, a character isolation or “segmentation” process may separate signal characters, for example character-based OCR algorithms. In some cases, a normalization process may normalize aspect ratio and/or scale of image component.

Still referring to FIG. 1, in some embodiments an OCR process will include an OCR algorithm. Exemplary OCR algorithms include matrix matching process and/or feature extraction processes. Matrix matching may involve comparing an image to a stored glyph on a pixel-by-pixel basis. In some case, matrix matching may also be known as “pattern matching,” “pattern recognition,” and/or “image correlation.” Matrix matching may rely on an input glyph being correctly isolated from the rest of the image component. Matrix matching may also rely on a stored glyph being in a similar font and at a same scale as input glyph. Matrix matching may work best with typewritten text.

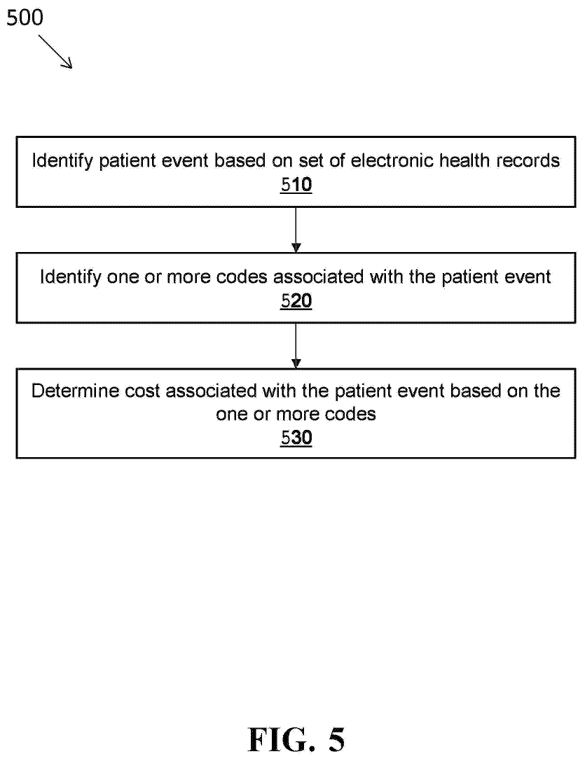

Still referring to FIG. 1, in some embodiments, an OCR process may include a feature extraction process. In some cases, feature extraction may decompose a glyph into features. Exemplary non-limiting features may include corners, edges, lines, closed loops, line direction, line intersections, and the like. In some cases, feature extraction may reduce dimensionality of representation and may make the recognition process computationally more efficient. In some cases, extracted feature can be compared with an abstract vector-like representation of a character, which might reduce to one or more glyph prototypes. General techniques of feature detection in computer vision are applicable to this type of OCR. In some embodiments, machine-learning process like nearest neighbor classifiers (e.g., k-nearest neighbors algorithm) can be used to compare image features with stored glyph features and choose a nearest match. OCR may employ any machine-learning process described in this disclosure, for example machine-learning processes described with reference to FIGS. 5-8. Exemplary non-limiting OCR software includes Cuneiform and Tesseract. Cuneiform is a multi-language, open-source optical character recognition system originally developed by Cognitive Technologies of Moscow, Russia. Tesseract is free OCR software originally developed by Hewlett-Packard of Palo Alto, California, United States.

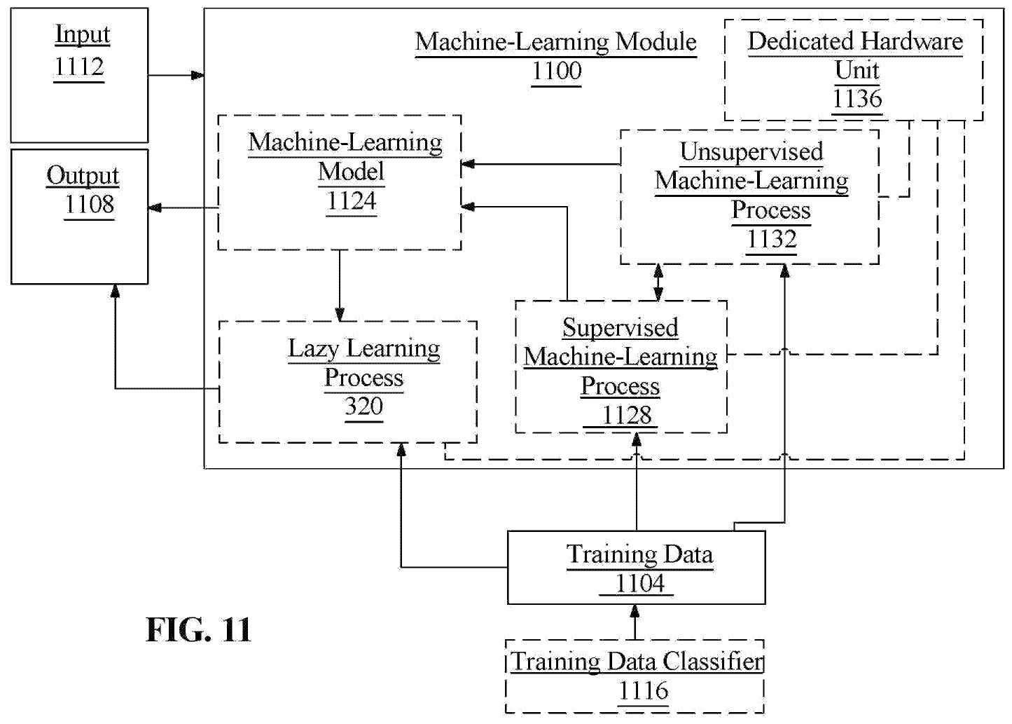

Still referring to FIG. 1, in some cases, OCR may employ a two-pass approach to character recognition. Second pass may include adaptive recognition and use letter shapes recognized with high confidence on a first pass to recognize better remaining letters on the second pass. In some cases, two-pass approach may be advantageous for unusual fonts or low-quality image components where visual verbal content may be distorted. Another exemplary OCR software tool include OCRopus. OCRopus development is led by German Research Centre for Artificial Intelligence in Kaiserslautern, Germany. In some cases, OCR software may employ neural networks, for example neural networks as taught in reference to FIGS. 11-13.

Still referring to FIG. 1, in some cases, OCR may include post-processing. For example, OCR accuracy can be increased, in some cases, if output is constrained by a lexicon. A lexicon may include a list or set of words that are allowed to occur in a document. In some cases, a lexicon may include, for instance, all the words in the English language, or a more technical lexicon for a specific field. In some cases, an output stream may be a plain text stream or file of characters. In some cases, an OCR process may preserve an original layout of visual verbal content. In some cases, near-neighbor analysis can make use of co-occurrence frequencies to correct errors, by noting that certain words are often seen together. For example, “Washington, D.C.” is generally far more common in English than “Washington DOC.” In some cases, an OCR process may make us of a priori knowledge of grammar for a language being recognized. For example, grammar rules may be used to help determine if a word is likely to be a verb or a noun. Distance conceptualization may be employed for recognition and classification. For example, a Levenshtein distance algorithm may be used in OCR post-processing to further optimize results.

With continued reference to FIG. 1, processor 108 is configured to retrieve auxiliary information 128 for the one or more vitality data sets 120. “Auxiliary information” for the purposes of this disclosure refers to supplementary data that enrich or support vitality data sets 120. For example, and without limitation, auxiliary information 128 may include the price of a particular medication listed within vitality data set 120. In one or more embodiments, auxiliary information 128 may include the price of medications within vitality data set 120, the price of treatment, the price of co-pays for medical visits, the price of medical procedures, usage data from medical devices, historical medical records, patients behavioral data not shown in an electronic health record and the like. In one or more embodiments, auxiliary information 128 include one or more quantitative data points 132. A “Quantitative data point” for the purposes of this disclosure is numerical information associated with at least one elements within vitality data set 120. For example, and without limitation, quantitative data point 132 may include the numerical price of a medication listed within vitality data set 120, the numerical price of a surgical procedure, the price of a doctor's visit, the price of a co-pay for a doctor's visit and the like. In one or more embodiments, while health records may contain information associated with the medical history of a patient, the records may not indicate the price of the services or medications given. In one or more embodiments quantitative data point 132 may include the overall cost of a procedure and/or an itemized list of costs. In one or more embodiments, auxiliary information 128 may include quantitative data points 132 wherein quantitative data points 132 may be associated with vitality data sets 120. In one or more embodiments, associations may include, but are not limited to associations with the cost of treatment. In one or more embodiments, auxiliary information 128 may further include missing elements 148. A “missing element” for the purposes of this disclosure is a portion of information that has been identified to be missing from vitality data set 120. For example, and without limitation, missing element 148 may include medication that seemingly may have been prescribed but is not indicated within vitality data set 120. Similarly, missing element 148 may include a particular surgical procedure that should have been present within vitality data set 120 yet was not present within vitality data set 120. In one or more embodiments, medical professionals and/or individuals associated with updating medical records may forget to insert and/or choose not to insert vital medical information. In an embodiment, auxiliary information 128 may allow for supplementation of the missing information to allow for a complete medical record to be present. In one or more embodiments, missing element 148 may include any information within vitality data set 120 that should be present but may be missing. In one or more embodiments, missing element 148 may further include any information needed to properly identify clusters 160 as described in further detail below. In one or more embodiments, missing element 148 may further include costs associated with medical treatment within vitality data set 120. In one or more embodiments, auxiliary information 128 may include any auxiliary information 128 and/or external variables as described in this disclosure.

With continued reference to FIG. 1, auxiliary information 128 may be received and/or generated. In one or more embodiments, auxiliary information 128 may be retrieved from database 116, wherein supplemental information may be received from database 116. In one or more embodiments, auxiliary informatio may be generated in any way as described in this disclosure. In one or more embodiments, auxiliary information 128 may be generated and/or received using an auxiliary module 136. An “auxiliary module” for the purposes of this disclosure is a system configured to receive vitality data sets 120 and output auxiliary information 128. In one or more embodiments, auxiliary module 136 may include any auxiliary information 128 generation module as described in this disclosure. In one or more embodiments, auxiliary module 136 may be configured to determine auxiliary information 128 needed and retrieve auxiliary information 128 as a result. For example, and without limitation, auxiliary module 136 may determine that a cost of a medication is missing and generate and/or retrieve the cost of the medication. In one or more embodiments, generation of auxiliary information 128 may include a two-step process wherein the required and/or missing information is first determined and then the information is received and/or generated. In one or more embodiments, auxiliary module 136 may be configured to receive one or more vitality data sets 120, determine at least one missing element 148 within one or more vitality data sets 120 and retrieve auxiliary information 128 as a function of missing element 148. In one or more embodiments, determining missing elements 148 may include the use of basic statical methods, data profiling, the use of checksums and the like. In one or more embodiments, in data profiling, processor 108 may analyze the patterns and distribution within a file in order to determine inconsistencies, outliers and potential gaps. In one or more embodiments, in checksums, processor 108 may identify missing or altered data by detecting checksum mismatches. In one or more embodiments, processor 108 may be configured to define data elements relevant to vitality data set 120 such as but not limited to patient demographics, medical history, and the like. In one or more embodiments, processor may then use the data elements to search through vitality data set 120 and determine and/or identify missing elements 148. In one or more embodiments, processor may utilize a null value check wherein processor 108 may determine whether a particular variable or field within vitality data set 120 is empty. In one or more embodiments, processor 108 may be configured to compare vitality data sets 120 to a plurality of parameters wherein each parameter may be used to identify a particular piece of relevant information. For example, and without limitation, a first parameter may be used to identify the patients name, the second may be used to identify medications, the third may be used to identify costs and the like. In an embodiments, failing to meet one or more parameters may indicate the identification of one or missing elements 148. In one or more embodiments, processor may be configured to first classify vitality data sets 120 using a classifier as described in this disclosure, wherein processor 108 may then be configured to identify missing elements 148 within each classified grouping. In one or more embodiments, processor 108 may be configured to use one or more data profiling tools to analyze trends and/or patterns within vitality data set 120 wherein processor 108 may be configured to determine anomalies in the patterns. In one or more embodiments, in an initial stage, processor 108 may be configured to receive a plurality of vitality data sets 120 and compare the plurality of vitality data sets to one another. In one or more embodiments, processor may determine “common element” wherein common elements may include data elements that occur in a significant number of vitality data sets 120. In one or more embodiments, processor 108 may then use common elements as a basis for determining missing elements 148 wherein a vitality data set 120 missing a common element may indicate that vitality data sets contains missing element 148. For example, and without limitation, a plurality of vitality data sets may indicate that a common element is patient demographics wherein a lack of patient demographics in one or more vitality data sets 120 may indicate a missing element 148.

With continued reference to FIG. 1, in one or more embodiments, patients notes, medication prescriptions, notes associated with a surgical procedure and the like may contain a predefined list of required information that is to be present. For example, and without limitation, a medication prescription may require the date of prescription wherein the date of prescription may be missing. In another non limiting example, a lab test result may be missing reference ranges wherein the reference ranges may indicate the suitable ranges for such results. In one or more embodiments, auxiliary module 136 may compare various records within vitality data set 120 to a predefined list of requirements wherein missing portions of those records may be labeled as missing elements 148. In one or more embodiments, in instances in which the records may be missing generic information, such as reference ranges, auxiliary module 136 may be configured to retrieve auxiliary information 128 associated with missing elements 148 from database 116. In one or more embodiments, in instances in which missing elements 148 may include prescription prices, processor 108 may be configured to retrieve missing elements 148 from database. In one or more embodiments, processor may use a patient's current diagnoses to predict various medications the patient may be taking in instances in which missing elements 148 includes medication information. In one or more embodiments, missing elements 148 may be input by a user, by the patient and the like. In one or more embodiments, missing elements 148 may be deduced from information contained within vitality data set. For example, and without limitation, information indicating age related disorders may be used to predict an age range for the patient. In another non limiting example, lab results showing abnormalities may be used to deduce that the patient may suffer from a particular condition not listed. In one or more embodiments, processor 108 may use a machine learning model such as any machine learning model as described in this disclosure to generate missing elements.

In one or more embodiments, database 116 may contain a plurality of quantitative data points 132 wherein each quantitative data point 132 may be associated with a particular medical procedure, a particular medication, a particular doctor's visit and the like. In one or more embodiments, auxiliary module 136 may be configured to identify various elements within vitality data set 120 that may require a quantitative data point 132 and search database 116 for the corresponding quantitative data point 132. For example, and without limitation, auxiliary module 136 may identify a medication such as ibuprofen, wherein database 116 may contain the price of ibuprofen. In one or more embodiments, database 116 may contain quantitative data points 132 categorized by service providers, pharmacies, insurance agencies and the like wherein each pharmacy, medical provider, medical insurance agency and the like may charge a set price for a particular medication or medical service. In one or more embodiments, insurance companies may have set co-pays wherein medications may be capped at a specific price. In one or more embodiments, auxiliary module 136 may be configured to determine an insurance provider of the patient as indicated within vitality data set 120 and provide estimated quantitative data points 132 based on the patient's co-pay amount. In one or more embodiments, a patient's medical insurance may indicate the costs that are due by the patient, in one or more embodiments, auxiliary module 136 may adjust prices and/or quantitative data points 132 based on the patient's out of pocket cost. For example, and without limitation, a patient may be required to pay 10% of a surgery at a maximum cost of 10,000$ a year wherein auxiliary module 136 may determine quantitative data points 132 based on the user's insurance. In one or more embodiments, quantitative data points 132 may vary with each insurance provider, wherein the presence of a particular insurance provider may indicate a particular quantitative data point 132. In one or more embodiments, auxiliary module 136 may determine quantitative data points 132 based on geographic location, the particular entity providing medical attention and the like. For example, and without limitation, a first pharmacy may charge a particular price for a first medication and a second pharmacy may charge a differing price. In one or more embodiments, quantitative data points 132 may be averaged based on geographic location wherein a particular geographic location, such as for example, Los Angeles, California may contain a differing average price as Orlando, Florida. In one or more embodiments, quantitative data points 132 may be input into database 116 by a user and retrieved by computing device 104. In one or more embodiments, quantitative data points 132 may be retrieved using a web crawler. In some embodiments, quantitative data points 132 may be derived from a web crawler. A “web crawler,” as used herein, is a program that systematically browses the internet for the purpose of Web indexing. The web crawler may be seeded with platform URLs, wherein the crawler may then visit the next related URL, retrieve the content, index the content, and/or measures the relevance of the content to the topic of interest. In some embodiments, computing device 104 may generate a web crawler to scrape quantitative data points 132 from a plurality of medical and/or pharmaceutical sites, blogs, or forums. The web crawler may be seeded and/or trained with a reputable website to begin the search. A web crawler may be generated by a processor 108. In some embodiments, the web crawler may be trained with information received from an external user through a user interface. In some embodiments, the web crawler may be configured to generate a web query. A web query may include search criteria received from a user. For example, a user may submit a plurality of websites for the web crawler to search for user data statistics from and correlate to elements of vitality data sets 120 to quantitative data elements. Additionally, the web crawler function may be configured to search for and/or detect one or more data patterns. A “data pattern” as used in this disclosure is any repeating forms of information. A data pattern may include repeating and/or alternating prices and the like. In some embodiments, the web crawler may be configured to determine the relevancy of a data pattern. Relevancy may be determined by a relevancy score. A relevancy score may be automatically generated by processor 108, received from a machine learning model, and/or received from the user. In some embodiments, a relevancy score may include a range of numerical values that may correspond to a relevancy strength of data received from a web crawler function. As a non-limiting example, a web crawler function may search the Internet for quantitative data points 132 related to an external user. The web crawler may return quantitative data points 132, such as, as non-limiting examples, prices for medications, prices for surgical procedures and the like.

With continued reference to FIG. 1, auxiliary module 136 may iteratively populate and update database 116 with new information received from WebCrawler, wherein information may be used following each iteration. In one or more embodiments, database 116 may be periodically and/or systematically updated independent of the current processing wherein each iteration of the processing may instead rely on data within database 116 instead of instantiating its own web crawler. In one or more embodiments, auxiliary module may utilize web crawler to retrieve auxiliary information 128. In one or more embodiments, processor 108 may populate a lookup table wherein quantitative data points 132 and/or auxiliary information 128 may be ‘looked up’ within the look-up table. A “lookup table,” for the purposes of this disclosure, is a data structure, such as without limitation an array of data, that maps input values to output values. A lookup table may be used to replace a runtime computation with an indexing operation or the like, such as an array indexing operation. A look-up table may be configured to pre-calculate and store data in static program storage, calculated as part of a program's initialization phase or even stored in hardware in application-specific platforms. In one or more embodiments, lookup table may be used to determine associated auxiliary information 128 for various elements within vitality data set 120. In one or more embodiments, processor 108 may determine the presence of elements requiring associated quantitative data points 132 by lookup up keywords using keyword recognition and/or pattern matching and using said key words within the lookup table to determine the associated quantitative data point 132. In one or more embodiments, processor 108 may be configured identify medications, medical procedures, medical treatments and the like using keyword recognition, pattern recognition and/or any other techniques in order to determine which elements may require quantitative data points 132.

With continued reference to FIG. 1, auxiliary module 136 may be configured to identify one or more treatment events 140. A “treatment event,” for the purposes of this disclosure refers to the process of a patient receiving medical treatment. For example, and without limitation, treatment event 140 may include the process of receiving treatment for an car infection wherein the process may include the initial doctor's visit, the notes written for the doctor's visit, the medication prescribed and/or the medication received. In another non limiting example, a treatment event 140 may include a surgical procedure wherein the surgical procedure may include the initial diagnostic procedures, various medical procedural treatments and various post procedural treatments. In an embodiment, each treatment event 140 may include a process to address a particular medical issue within a particular time frame. For example, and without limitation, a treatment event 140 may include a 4 day span in which a patient is in the hospital to receive treatment for a medical condition. In another non limiting example, treatment event 140 may include a routine doctors visit wherein an information within vitality data set 120 associated with the routine visit may be associated with the treatment event 140. In one or more embodiments, treatment events 140 may include surgical procedures, medical visits, and the like. In one or more embodiments, treatment events 140 may include any patient events and/or medication events as described in this disclosure. In one or more embodiments, treatment event 140 may include any information associated with a doctor's visit, any medical treatment given on a single day, any information associated with similar medical treatments and procedures and the like. In one or more embodiments, treatment events 140 may include grouping of data wherein each grouping is associated with a particular treatment, particular day and the like.

With continued reference to FIG. 1, processor 108 may be configured to identify one or more treatment by classifying information within vitality data set 120 to one or more treatment events 140. In an embodiments, treatment events 140 may include groupings of information categorized by a particular data, a particular medical issue being treated and the like. In one or more embodiments, processor 108 may use a classifier to classify elements of vitality data set 120 to one or more treatment events 140. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. Classifiers as described throughout this disclosure may be configured to output at least a datum that labels or otherwise identifies a set of data that are clustered together, found to be close under a distance metric as described below, or the like.

With continued reference to FIG. 1, processor 108 108 may be configured to generate classifiers as described throughout this disclosure using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters 160 and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database 116, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process for the purposes of this disclosure. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors algorithm may generate a first vector output containing a data entry cluster 160, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors for the purposes of this disclosure may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where αᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With continued reference to FIG. 1, processor 108 may classify vitality data set 120 to one or more treatment events 140. In one or more embodiments, identification of one or more treatment events 140 may include classification of vitality dataset to one or more treatment events 140. In one or more embodiments, processor 108 may use a treatment classifier wherein the treatment classifier may be configured to classify data elements within vitality data set 120 to one or more treatment events 140. In one or more embodiments, classifiers as described throughout this disclosure may be configured to output at least a datum that labels or otherwise identifies a set of data that are clustered together, found to be close under a distance metric as described below, or the like. In some cases, processor 108 may generate, and train treatment classifier configured to receive vitality data set 120 and output one or more treatment events 140. Processor 108 and/or another device may generate a classifier using a classification algorithm, defined as a process whereby a computing device 104 derives a classifier from training data. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers. Treatment classifier may be trained with training data correlating vitality data sets 120 to one or more treatment events 140. Training data may include a plurality of vitality data sets 120 correlated to a plurality of treatment events 140. In an embodiment, training data may be used to show that a particular element or elements within vitality data set 120 may be correlated to a particular treatment event 140. Training data may be received from an external computing device 104, input by a user, database 116 and/or previous iterations of processing. Treatment classifier may be configured to receive as input and categorize components of vitality data set 120 to one or more treatment events 140. In some cases, processor 108 may then select any elements within vitality data set 120 containing a similar label and/or grouping and group them together. In some cases, vitality data set 120 may be classified using a classifier machine learning model 152 and/or a classifier. In some cases, classifier machine learning model 152 may be trained using training data correlating a plurality of vitality data sets 120 correlated to a plurality of treatment events 140. In an embodiment, a particular element within vitality data set 120 may be correlated to a particular treatment event 140. In some cases, classifying vitality data set 120 may include classifying vitality data set 120 as a function of the classifier machine learning model 152. In some cases classifier training data may be generated through input by a user. In some cases, classifier machine learning model 152 may be trained through user feedback wherein a user may indicate whether a particular element corresponds to a particular categorization and/or treatment event 140. In some cases, classifier machine learning model 152 may be trained using inputs and outputs based on previous iterations. In some cases, a user may input previous vitality data sets 120 and corresponding treatment events 140 wherein classifier machine learning model 152 may be trained based on the input.

With continued reference to FIG. 1, in some embodiments, classifier training data may be iteratively updated using feedback. Feedback, in some embodiments, may include user feedback. For example, user feedback may include a rating, such as a rating from 1-10, 1-100, −1 to 1, “happy,” “sad,” and the like. In some embodiments, user feedback may rate a user's satisfaction with the classification of one or more elements to a treatment event 140. In one or more embodiments, classifier machine learning model 152 may be iteratively trained wherein a suer may provide feedback indicating if inputs contain the correct correlated outputs.

With continued reference to FIG. 1, identification of one or more treatment events 140 may be done through keyword recognition wherein data files containing similar information may be grouped together. For example, data files containing information indicating that the data files correspond to a particular treatment may be grouped together. In one or more embodiments, identifying treatment event 140 may include generating a timeframe 144 for each treatment event 140. A “timeframe,” for the purposes of this disclosure is a determined duration for a treatment event 140. For example, and without limitation, time frame may include the span of a day, the span of an hour, the span of a week and the like. In an embodiments, each treatment event 140 may include a time frame wherein the time frame may indicate how long the treatment event 140 took. In one or more embodiments, processor 108 may generate timeframes 144 by receiving dates and times located on files within vitality data set 120. In an embodiments, each document, note and the like may contain a time and date stamp wherein similar time and date stamps may be used to identify treatment events 140. For example, and without limitation, two data files and/or digital files dates for the same day may be associated with the same treatment event 140. In one or more embodiments, timeframes 144 may be generated by aggregating times and times within each vitality data set 120 and grouping similar dates and times, such as those on the same day, those on consecutive days and the like. In an embodiments, two data files corresponding to the same medical treatment and recorded on consecutive days may indicate that two data files are within the same timeframe 144 and resultingly, two data files are associated with the same treatment event 140. In one or more embodiments, processor 108 may generate a timeframe 144 by extracting dates and times within vitality data set 120 and grouping dates and times that are similar, such as for example, similar dates, consecutive dates and the like.

With continued reference to FIG. 1, processor 108 may be configured to generate auxiliary information 128 for each treatment event 140. In one or more embodiments, processor 108 may be configured to group information within vitality data set 120 to one or more treatment events 140 and generate auxiliary information 128 for each treatment event 140. In one or more embodiments, processor 108 may be configured to retrieve one or more quantitative data points 132 for each treatment event 140 wherein elements within each treatment event 140 having associated quantitative data points 132 may be associated with treatment event 140. In one or more embodiments, grouping of treatment events 140 may allow for processor 108 and/or a user to determine which quantitative data points 132 may be associated with a particular treatment event 140. For example, and without limitation, a quantitative data point 132 such a cost for a surgical procured may be determined to be classified to and/or categorized within a surgical procedure event. In an embodiment, processor 108 may be configured to retrieve quantitative data points 132 for each treatment event 140 and group the quantitative data points 132. In one or more embodiments, processor 108 may be configured to group quantitative data points 132 based on relative times frames wherein elements and/r files within given times frames may contain associated quantitative data points 132 that may be grouped. In one or more embodiments, quantitative data points 132 may be used to determine the overall cost of a procedure and/or treatment event 140. In one or more embodiments, processor 108 may group quantitative data points 132 within each treatment event 140 in order to determine costs associated with treatment events 140. In one or more embodiments, processor 108 may sum up quantitative data points 132 to determine overall costs associated with a treatment event 140. For example, and without limitation, processor 108 may determine that the overall costs associated with a treatment event 140 may be 100$, wherein 50$ may be associated with medication and 50$ may be associated with the doctor's visit. In one or more embodiments, quantitative data points 132 may be used to quantify one or more elements within vitality data set. In one or more embodiments, quantitative data points 132 may be used to quantify one or more elements wherein processor 108 may be configured to determine costs associated with medication, treatment and the like. In an embodiments, vitality data set 120 may include a plurality of elements wherein each element may refer to a small portion of vitality data set 120. In one or more embodiments, elements may refer to medications, treatment events 140, doctor visits and the like. In one or more embodiments, quantitative data points 132 may be used to quantify elements in order to determine costs associated with one or more elements within vitality data ser 120.

With continued reference to FIG. 1, processor 108 may use a machine learning model such as any machine learning model as described in this disclosure in order to retrieve and/or generate auxiliary information 128 and/or quantitative data points 132. In one or more embodiments, processor 108 may utilize an auxiliary machine learning model to receive vitality data sets 120 and output auxiliary information 128. “Auxiliary machine learning model” for the purposes of this disclosure is a machine learning model configured to receive multiple inputs and determine correlated auxiliary information 128 for the inputs. Auxiliary machine learning model may receive inputs such as vitality data set 120 and/or a plurality thereof and determine auxiliary information 128 for the vitality data set 120. In an embodiments, an element within vitality data set 120 may include a correlated quantitative data point 132. In yet another embodiments, auxiliary machine learning model may be configured to retrieve missing elements 148 within vitality data set 120. In one or more embodiments, auxiliary machine learning model may include any machine learning model as described in this disclosure. In one or more embodiments, an auxiliary machine learning module may be configured to create auxiliary machine learning model. In one or more embodiments, auxiliary machine learning model may include a neural network, a supervised machine learning model and/or any other machine learning model as described in this disclosure. In one or more embodiments auxiliary machine learning model may be trained with auxiliary training data. In one or more embodiments, auxiliary training data may include a plurality of vitality data sets 120 correlated to a plurality of auxiliary information 128. In an embodiments, a particular vitality data set 120 and/or element thereof may be associated with an output of auxiliary information 128. In one or more embodiments, auxiliary machine learning model may be used to output associated costs of a medical treatment, to determine missing elements 148 and the like. In one or more embodiment auxiliary training data may be generated by a user, 3^{rd} party and the like. In one or more embodiments, auxiliary training data may be received from a user, database 116 and the like. In one or more embodiments, auxiliary training data may include vitality data sets 120 and correlated auxiliary information 128 of previous iterations. In one or more embodiments, vitality training data may be iteratively updated with vitality data sets 120 from current iterations and correlated auxiliary information 128. In one or more embodiments, auxiliary machine learning model may be iteratively trained using data from current iterations. In one or more embodiments, training may include vitality data sets 120 and correlated auxiliary information 128 that have received user feedback. In one or more embodiments, user feedback may be used to train auxiliary machine learning model wherein user feedback may be used to indicate if a particular missing element 148 and/or cost was correct. In one or more embodiments, auxiliary training data may be classified based on treatment events 140 wherein vitality data set 120 classified to a treatment event 140 may be input and auxiliary information 128 classified to the treatment event 140 may be output. In an embodiments, classification of vitality data set 120 and/or auxiliary training data may allow for more accurate results wherein outputs may be classified to the same inputs.

With continued reference to FIG. 1, processor 108 is configured to generate one more augmented data set 156 as a function of the auxiliary information 128 and the one or more vitality data sets 120. An “augmented data set,” for the purposes of this disclosure is information within vitality data set 120 and the corresponding auxiliary information 128 in a text format. For example, and without limitation, vitality data set 120 may include digital documents that have not been scanned, wherein augmented data set 156 may include textual data of the digital documents as well the retrieved auxiliary information 128. In one or more embodiments, augmented data set 156 may include vitality data sets 120 including information that had been indicated to be missing within vitality data sets 120, such as auxiliary information 128 and/or missing elements 148. In an embodiments, processor may be used to aggregate missing elements 140, auxiliary information 128 and/or vitality data set 120 to create augmented data set 156. In one or more embodiments, augmented data set 156 may include vitality data set 120 with any corresponding information needed for processing. In one or more embodiments, one or more elements within vitality data set 120 may be missing wherein augmented data set 156 may include the missing information. In one or more embodiments augmented data set 156 may include quantitative data points 132 and/or costs associated with various elements with vitality data set 120. In one or more embodiments, processor 108 may generate augmented data set 156 by aggregating vitality data set 120 and auxiliary information 128. In one or more embodiments, processor 108 may generate augmented data set 156 by aggregating textual data from vitality data set 120 and auxiliary information 128In one or more embodiments, textual data may be aggregated using optical character recognition wherein textual data may be produced from digital files. In one or more embodiment, vitality data set may include a mixture of textual data and digital files wherein OCR may be used to convert any remaining digital files into textual data. In one or more embodiment, processor may be configured to combine the textual data with auxiliary information to create augmented data set 156. In one or more embodiments, augmented data set 156 may include information within vitality data set 120 categorized into groupings wherein auxiliary information 128 may be placed into their corresponding groupings. In one or more embodiments, augmented data set 156 may include augmented health records such as any augmented health records and/or electronic health records as described in this disclosure.

With continued reference to FIG. 1, processor 108 is configured to identify one cluster 160 based on the one or more augmented data sets 156. A “cluster,” for the purposes of this disclosure is a grouping of augmented data sets 156 based on similar medical and/or biological characteristics. For example, and without limitation, patients within a similar age range may be placed within a first cluster 160 and patients with a particular medical history may be placed within a second cluster 160. In one or more embodiments, clusters 160 may include but are not limited, groupings such as groupings associated with age, gender, medical history, current medical diagnosis, financial background, medical treatments received, height, weight, physical activity, cost of medication paid and the like. In one or more embodiments, each augmented data set 156 of a plurality of augmented data sets 156 contain similar categories of information such as but not limited to age, medical background and the like wherein each category may be used to determine clusters 160. In one or more embodiments, clusters 160 may include and/or be included within cohorts such as any cohorts as described in this disclosure. In one or more embodiments, clusters 160 may be identified, and/or generated in any way as described in this disclosure similar to that of cohorts as described in further detail below. In one or more embodiments, processor 108 may determine clusters 160 by comparing elements within augmented data sets 156 and aggregating augmented data sets 156 with similar elements. For example, and without limitation, processor 108 may create a cluster 160 of patients with a particular neurological disorder wherein cluster 160 may include individuals with the same neurological disorder. In one or more embodiments, each cluster 160 may include one or more augmented data sets 156. In one or more embodiments, augmented data sets 156 may be classified to more than one clusters 160. In one or more embodiments, processor 108 may be configured to generate subclusters, wherein subclusters include groupings within clusters 160. For example, and without limitation, processor 108 may be configured to generate a grouping of people with a particular medical condition and a subgrouping of people within a particular age range with the medical condition. In another non limiting example, processor 108 may be configured to generate a grouping of people with a first particular medical condition and a subgrouping of people with a second similar medical condition. In one or more embodiments, processor 108 may be configured to link up people with similar age ranges such as for example, 15-20,20-25,60-65,70-75 and the like. In one or more embodiments, processor 108 may be configured to generate clusters 160 based on two or more similarities, wherein for example, augmented data sets 156 associated with a similar gender and a similar medical condition may be created into cluster 160. In one or more embodiments, clusters 160 may be generated based on financial status of patients within augmented data sets 156, costs paid as indicated by quantitative data points 132 and the like. In one or more embodiments, subclusters may be used to determine similarities and/or indications that may have contributed and/or exacerbated a particular medical condition. For example, and without limitation, a subcluster of individuals paying an exorbitant amount for medical treatment may indicate that the high price of medical treatment may have caused an individual not to seek medical attention. In one or more embodiments, clusters 160 may be generated using classifiers wherein augmented data sets 156 may be classified to clusters 160. In one or more embodiments, augmented data sets 156 may be classified to clusters 160 using classifier machine learning model 152 such as classifier machine learning model 152 as described above. In one or more embodiments, classifier machine learning model 152 may receive augmented data sets 156 and outputs clusters 160. In one or more embodiments, classifier machine learning model 152 may receive augmented data set 156 and output clusters 160. In one or more embodiments, classifier machine learning model 152 may be trained with classifier training data. In one or more embodiments, classifier training data may include a plurality of augmented data sets 156 correlated to a plurality of clusters 160. In one or more embodiment, classifier machine learning model 152 may be used to find similarities between a given set of augmented data sets 156 and group augmented data sets 156 into clusters 160. In one or more embodiments, classifier machine learning model 152 may be iteratively trained wherein a user may provide feedback based on correctly and wrongfully placed augmented data sets 156 within clusters 160. In one or more embodiments, current iterations of augmented data set 156 may be used to iteratively train classifier machine learning model 152. In one or more embodiments, processor 108 may identify clusters 160 by comparing similar elements within each augmented data set 156. For example, and without limitation, processor 108 may compare medical conditions, financial backgrounds, biological backgrounds, medications taken, medications paid and the like and group similar medications, similar procedures, similar backgrounds and the like. In one or more embodiments, identifying clusters 160 may include any processor 108 and/or determination wherein identifying cohorts as described in further detail below.

With continued reference to FIG. 1, in one or more embodiments, clusters 160 may be used to determine similarities and differences between patients. In an embodiments, comparing patients within similar age ranges, medical conditions and the like may provide insights such as similarities in individuals with a particular condition. In one more embodiments, clusters 160 may be used to predict medical conditions, medical treatment, and the like wherein an individual may be determined to have a particular disease and/or more susceptible to a particular disease based on their classification to a cluster 160.

With continued reference to FIG. 1, processor 108 is configured to provide cluster 160 to a cluster analysis platform 164. A “cluster analysis platform” for the purposes of this disclosure is a system used to find similarities or trends between vitality data sets 120 within clusters 160. For example, and without limitation, cluster analysis platform 164 may be used to determine an individual's propensity to being diagnosed with a disease. In another non limiting example, cluster analysis platform 164 may be used to determine the roots of a particular medical condition due to trends seen in clusters 160. In one or more embodiments, cluster analysis platform 164 may include a cohort analysis platform as described in further detail below. In one or more embodiments, clusters 160 may be analyzed and/or determined in any way similar to that of cohorts as described in further detail below. In one or more embodiments, cluster analysis platform 164 may include a graphical user interface wherein the graphical user interface may allow a user to view clusters 160 and make determinations based on the clusters 160. In one or more embodiments, cluster analysis platform 164 may be used to train auxiliary machine learning model to detect missing elements 148. In an embodiments, a complete health record may be generated for each patient as indicated by augmented health record. In an embodiments, augmented health records may be fed into auxiliary machine learning model wherein missing elements 148 from future vitality data sets 120 may be determined. In one or more embodiments, missing elements 148 may be determined and/or predicted based on classification to a particular cluster 160.

With continued reference to FIG. 1, cluster analysis platform 164 may be configured to generate similarity datum 180 as a function of the vitality data set and clusters. “Similarity datum” for the purposes of this disclosure is information indicating similarities or trends between one or more vitality data sets 120 within a cluster 160. For example, and without limitation, similarity datum 180 may indicate that a cluster 160 associated with patients over ages 65 may all have similarities such that they are all more prone to heart attacks. In another non limiting example, similarity datum 180 may indicate that a cluster of patients with diabetes all have cardiovascular issues and/or may tend to have cardiovascular issues. In one or more embodiments, similarity datum 180 may indicate potential trends and/or similarities wherein similar datum may indicate people within a particular cluster 160 may be more prone to a particular diseases. In one or more embodiments, similarity datum 180 may include trends wherein patients classified to a particular cluster may exhibit similar trends in their health increasing or declining. In one or more embodiments, similarity datum 180 may include possible correlations between clusters and health conditions wherein a particular cluster having similar health conditions may be attributed to similar factors. For example, and without limitation, similarity datum 180 may include correlations between poverty and health wherein individuals who could not afford a particular medication had a lower health outcome. In one or more embodiments, clusters 160 may be used to group patients and/or vitality data sets within similar poverty levels, clinical cohorts and the like wherein similarity datum 180 may include insights and/or similarities that may indicate causation rather than correlation. In one or more embodiments, absent auxiliary information, such as prescription costs, some correlations or causations cannot be made and/or may be inaccurate. For example, and without limitation, it may be through that a particular disease state may be the result of an individual's health whereas auxiliary information 128 may be used to indicate that the cost of the medication may have been the result. In an embodiment, comparison of vitality data sets 120 alone may create inaccurate correlations or causations wherein comparison of augmented data sets 156 may increase the accuracy of similarities. In one or more embodiments, cluster analysis platform 164 may compare elements within augmented data sets 156 to determine common elements (as described above) and generate similarity using common elements. For example, without limitation cluster analysis platform 164 may determine common elements such as a particular disease within a cluster 160 wherein similarity datum 180 may include the disease. In one or more embodiments, similarity datum 180 may include similar trends within patients. For example, and without limitation, similarity datum 180 may indicate that patients who cannot afford their medication are more likely to have more severe health effects. In one or more embodiments, similarity datum 180 may include trends over a given particular time frame wherein similarity datum 180 may indicate patients within similar clusters may be affected by similar diseases over a given time frame. In one or more embodiments, similarity datum 180 may include similarities within portions of each cluster, wherein a grouping of people within a cluster may contain similarities and another separate grouping within the same cluster 160 may share differing similarities. In one or more embodiments, similarity datum 180 may be used to indicate which medications within a cluster were effective, which medications were effective, the types of people within a cluster 160 most affected and the like. In one or more embodiments, cluster analysis platform 164 may make determinations within clusters 160 In one or more embodiments, cluster analysis platform 164 may be configured to generate similarity datum 180 wherein similarity datum 180 may include information associated with subclusters. In an embodiments, similarities within subclusters may be used to determine key differences and outcomes between individuals within a cluster 160. In one or more embodiments, similarity datum 180 including information about subclusters may be used to determine what medications were affected, how a particular group of individuals differ from another group within the same cluster 160, how particular similarities affected a subcluster and the like. In one or more embodiments, similarity datum 180 may be generated by comparing augmented data sets 156 within similar clusters and/or sub clusters. In one or more embodiments, cluster analysis platform 164 may utilize one or more machine learning models to generate similarity datum 180. In one or more embodiments, the machine learning models may be configured to receive augmented data sets 156 and determine similarities between various augmented data sets within a cluster 160.

With continued reference to FIG. 1, similarity datum 180 may include outlier data. “Outlier data” for the purposes of this disclosure is information within an augmented data that lacks similarity to other augmented data sets within a cluster. For example, and without limitation, outlier data may indicate that a particular individual took a medication that differed from the rest of the cluster, an individual had a differing treatment option than others in a cluster 160, an individual had a differing treatment cost in comparison to others in a cluster 160 and the like. In an embodiments, outlier data may be used to filter out individuals who may cause results to skew. In one or more embodiments, outlier data may be used to determine how differences between individuals may cause changes to occur. In one or more embodiments, outlier data may be used to determine if a particular element within augmented data set was the cause of a particular issue. For example, and without limitation, outlier data indicating that an individual took a lower or higher dosing of a particular medication yet still achieved similar results may indicate that dosing was not a contributing factor in the overall health within the cluster 160. Similarly, an individual who took additional differing medication in comparison to others within a cluster yet saw no significant differences in comparison to others may indicate that the additional medication was not effective. In contrast, outlier data may also be used to determine if changes within one augmented data set 156 may have contributed to other factors. For example, and without limitation, an increase in dosage of a medication within an individual which resulted in an increase in health may indicate that the additional dosing may be effective.

With continued reference to FIG. 1, cluster analysis platform 164 may utilize correlation analysis process to determine relationships between variables within augmented data sets 156 to generate similarity datum 180. In one or more embodiments, cluster analysis platform may use clustering algorithms to determine similarities between augmented data sets 156. A feature learning and/or clustering algorithm may be implemented, as a non-limiting example, using a k-means clustering algorithm. A “k-means clustering algorithm” as used in this disclosure, includes cluster analysis that partitions n observations or unclassified cluster data entries into k clusters in which each observation or unclassified cluster data entry belongs to the cluster with the nearest mean. “Cluster analysis” as used in this disclosure, includes grouping a set of observations or data entries in way that observations or data entries in the same group or cluster are more similar to each other than to those in other groups or clusters. Cluster analysis may be performed by various cluster models that include connectivity models such as hierarchical clustering, centroid models such as k-means, distribution models such as multivariate normal distribution, density models such as density-based spatial clustering of applications with nose (DBSCAN) and ordering points to identify the clustering structure (OPTICS), subspace models such as biclustering, group models, graph-based models such as a clique, signed graph models, neural models, and the like. Cluster analysis may include hard clustering whereby each observation or unclassified cluster data entry belongs to a cluster or not. Cluster analysis may include soft clustering or fuzzy clustering whereby each observation or unclassified cluster data entry belongs to each cluster to a certain degree such as for example a likelihood of belonging to a cluster; for instance, and without limitation, a fuzzy clustering algorithm may be used to identify clustering of elements of a first type or category with elements of a second type or category, and vice versa. Cluster analysis may include strict partitioning clustering whereby each observation or unclassified cluster data entry belongs to exactly one cluster. Cluster analysis may include strict partitioning clustering with outliers whereby observations or unclassified cluster data entries may belong to no cluster and may be considered outliers. Cluster analysis may include overlapping clustering whereby observations or unclassified cluster data entries may belong to more than one cluster. Cluster analysis may include hierarchical clustering whereby observations or unclassified cluster data entries that belong to a child cluster also belong to a parent cluster.

With continued reference to FIG. 1, computing device 104 may generate a k-means clustering algorithm receiving unclassified data and outputs a definite number of classified data entry clusters wherein the data entry clusters each contain cluster data entries. K-means algorithm may select a specific number of groups or clusters to output, identified by a variable “k.” Generating a k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, this may be utilized to select a k value. Centroids of k-groups or k-cluster include a collection of feature values which are utilized to classify data entry clusters containing cluster data entries. K-means clustering algorithm may act to identify clusters of closely related data, which may be provided with user cohort labels; this may, for instance, generate an initial set of user cohort labels from an initial set of data, and may also, upon subsequent iterations, identify new clusters to be provided new labels, to which additional data may be classified, or to which previously used data may be reclassified.

With continued reference to FIG. 1, computing device may generate a k-means clustering algorithm receiving unclassified data and outputs a definite number of classified data entry clusters wherein the data entry clusters each contain cluster data entries. K-means algorithm may select a specific number of groups or clusters to output, identified by a variable “k.” Generating a k-means clustering algorithm includes assigning inputs containing unclassified data to a “k-group” or “k-cluster” based on feature similarity. Centroids of k-groups or k-clusters may be utilized to generate classified data entry cluster. K-means clustering algorithm may select and/or be provided “k” variable by calculating k-means clustering algorithm for a range of k values and comparing results. K-means clustering algorithm may compare results across different values of k as the mean distance between cluster data entries and cluster centroid. K-means clustering algorithm may calculate mean distance to a centroid as a function of k value, and the location of where the rate of decrease starts to sharply shift, this may be utilized to select a k value. Centroids of k-groups or k-cluster include a collection of feature values which are utilized to classify data entry clusters containing cluster data entries. K-means clustering algorithm may act to identify clusters of closely related data, which may be provided with user cohort labels; this may, for instance, generate an initial set of user cohort labels from an initial set of data, and may also, upon subsequent iterations, identify new clusters to be provided new labels, to which additional data may be classified, or to which previously used data may be reclassified.

With continued reference to FIG. 1, generating a k-means clustering algorithm may include generating initial estimates for k centroids which may be randomly generated or randomly selected from unclassified data input. K centroids may be utilized to define one or more clusters. K-means clustering algorithm may assign unclassified data to one or more k-centroids based on the squared Euclidean distance by first performing a data assigned step of unclassified data. K-means clustering algorithm may assign unclassified data to its nearest centroid based on the collection of centroids ci of centroids in set C. Unclassified data may be assigned to a cluster based on argmin_{ci} [symbol] _{C}dist (ci, x)², where argmin includes argument of the minimum, ci includes a collection of centroids in a set C, and dist includes standard Euclidean distance. K-means clustering module may then recompute centroids by taking mean of all cluster data entries assigned to a centroid's cluster. This may be calculated based on ci=1/|Si|Σxi [symbol] Si^{xi}. K-means clustering algorithm may continue to repeat these calculations until a stopping criterion has been satisfied such as when cluster data entries do not change clusters, the sum of the distances have been minimized, and/or some maximum number of iterations has been reached.

Still referring to FIG. 1, k-means clustering algorithm may be configured to calculate a degree of similarity index value. A “degree of similarity index value” as used in this disclosure, includes a distance measurement indicating a measurement between each data entry cluster generated by k-means clustering algorithm and a selected element. Degree of similarity index value may indicate how close a particular combination of elements is to being classified by k-means algorithm to a particular cluster. K-means clustering algorithm may evaluate the distances of the combination of elements to the k-number of clusters output by k-means clustering algorithm. Short distances between an element of data and a cluster may indicate a higher degree of similarity between the element of data and a particular cluster. Longer distances between an element and a cluster may indicate a lower degree of similarity between elements to be compared and/or clustered and a particular cluster.

With continued reference to FIG. 1, k-means clustering algorithm selects a classified data entry cluster as a function of the degree of similarity index value. In an embodiment, k-means clustering algorithm may select a classified data entry cluster with the smallest degree of similarity index value indicating a high degree of similarity between an element and the data entry cluster. Alternatively or additionally k-means clustering algorithm may select a plurality of clusters having low degree of similarity index values to elements to be compared and/or clustered thereto, indicative of greater degrees of similarity. Degree of similarity index values may be compared to a threshold number indicating a minimal degree of relatedness suitable for inclusion of a set of element data in a cluster, where degree of similarity indices a-n falling under the threshold number may be included as indicative of high degrees of relatedness. The above-described illustration of feature learning using k-means clustering is included for illustrative purposes only and should not be construed as limiting potential implementation of feature learning algorithms; persons skilled in the art, upon reviewing the entirety of this disclosure, will be aware of various additional or alternative feature learning approaches that may be used consistently with this disclosure.

With continued reference to FIG. 1, cluster analysis platform 164 may use cluster algorithms as described above to generate similarity datum 180. In one or more embodiments, cluster analysis platform may use hashing to generate has values for elements within augmented data sets 156 and determine similarities. In one or more embodiments, cluster analysis platform may use a machine learning model such as nay machine learning model to create similarity datum 180. In an embodiment, simple comparison processes may not be able to find similarities between augmented data sets due to the use of differing words, patterns, techniques and the like used by different providers. In an embodiment, cluster analysis platform may use a machine learning model, such as similarity machine learning model to generate similarity datum 180. In one or more embodiments, similarity machine learning model may include any machine learning model as described in this disclosure. In one or more embodiments, similarity machine learning model may be configured to find similarities within augmented data sets 156 in instances in which simple element or word matching may not suffice. In one or more embodiments, similarity machine learning model may be configured to identify medications, diseases states, costs and the like and determine similarities between augmented data sets 156. In one or more embodiments, similarity machine learning model may be configured to receive as an input, augmented data sets and output words or phrases that may be used to summate disease states, ages, medications and the like. In one or more embodiments, the output words or phrases may be compared by processes to determine similarities between augmented data sets 156. In one or more embodiments, similarity machine learning model may be configured to receive augmented data sets 156 and output similarity datum 180. In one or more embodiments, similarity machine learning model may be configured to receive augmented data set 156 and output keywords or phrases for each augmented data set 156 wherein processor 108 may be configured to compare the keywords or phrases to determine similarities. In an embodiment, an output of keywords or phrases may allow for increased computational efficiency wherein machine learning models may be used for more complex tasks whereas less complex tasks such as data matching may be used through simpler comparison processes.

With continued reference to FIG. 1, in one or more embodiments, cluster analysis platform 164 may include a user interface such as a graphical user interface. In one or more embodiments, processor 108 may be configured to generate a graphical user interface for cluster analysis platform 164 by creating a user interface data structure. As used in this disclosure, “user interface data structure” is a data structure representing a specialized formatting of data on a computer configured such that the information can be effectively presented for a user interface. User interface data structure may include clusters 160, vitality data sets 120, auxiliary information 128, similarity datum 180, and the like. In one or more embodiments, graphical user interface may be configured to present similarity datum 180 within a graphical format. A graphical format” for the purposes of this disclosure is a visual representation of textual information. For example, and without limitation, textual information such as numerical values may be plotted on an X-Y chart in order to show data within a graphical format. In one or more embodiments, graphical format mat may allow for visualization of textual data such as similarity datum 180 in order to view similarity datum 180 through graphical user interface. In one or more embodiments, graphical format may include a similarity matrix wherein similarity matrix may include a color coded matrix. In one or more embodiments, the color coded matrix may contain colors for each degree of similarity (e.g. elements common in most or all augmented data sets 156) wherein higher degrees of similarity may be given one color and lower degrees of similarity may be another color. For example, and without limitation, similarity matrix may contain a plurality of cells wherein each cell contains an element of similarity datum 180. Continuing, in instances in which the element is most commonly shared, the cell may be color coded to green, wherein less common elements may be yellow and elements having little to no commonality may be red. In one or more embodiments, graphical format may include X-Y charts showing trends within particular clusters 160 such as but not limited to, trends in health decline, trends in weight loss, trends in health increase and the like. In one or more embodiments, similarity datum 180 may be used to compare or contrast multiple clusters 160 and/or subclusters wherein graphical format may visualize differences between clusters. For example and without limitation, graphical format may include color coded data showing health associated with a particular medication in one cluster and health associated with a differing medication in another cluster. In one or more embodiments, processor 108, cluster analysis platform 164 and/or graphical user interface may be configured to display similarity datum 180 in graphical format. In one or more embodiments, similarities within clusters 160 may be mapped over a given period of time, such as day month year, several years and the like. In one or more embodiments, graphical formats may include charts depicting changes in health, changes in medication, changes in disease states and the like over time. Inn one or more embodiments, processor 108 and/or cluster analysis platform may be configured to map individual's health or diseases states over time for each cluster to determine how each particular cluster may have contributed to the increase or decline in a groups' health. In one or more embodiments, graphical format may include heatmaps which may visualize similarities within clusters such as subclusters.

With continued reference to FIG. 1, processor 108 may be configured to transmit the user interface data structure to a graphical user interface. Transmitting may include, and without limitation, transmitting using a wired or wireless connection, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. Processor 108 may transmit the data described above to database 116 wherein the data may be accessed from database 116. Processor 108 may further transmit the data above to a device display or another computing device 104.

With continued reference to FIG. 1, system may include a graphical user interface (GUI 168). For the purposes of this disclosure, a “user interface” is a means by which a user and a computer system interact. For example, through the use of input devices and software. In some cases, processor 108 may be configured to modify graphical user interface as a function of the distortion outputs by populating user interface data structure and visually presenting the data through modification of the graphical user interface. A user interface may include graphical user interface, command line interface (CLI), menu-driven user interface, touch user interface, voice user interface (VUI), form-based user interface, any combination thereof and the like. In some embodiments, a user may interact with the user interface using a computing device 104 distinct from and communicatively connected to processor 108. For example, a smart phone, smart tablet, or laptop operated by the user and/or participant. A user interface may include one or more graphical locator and/or cursor facilities allowing a user to interact with graphical models and/or combinations thereof, for instance using a touchscreen, touchpad, mouse, keyboard, and/or other manual data entry device. A “graphical user interface,” as used herein, is a user interface that allows users to interact with electronic devices through visual representations. In some embodiments, GUI 168 may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in graphical user interface. Persons skilled in the art, upon reviewing the entirety of this disclosure, will be aware of various ways in which a graphical user interface and/or elements thereof may be implemented and/or used as described in this disclosure. In one or more embodiments, graphical user interface may include a graphical charts depicting vitality data sets 120. In one or more embodiments, quantitative elements such as numbers, may be charted in order to graphically view differences between vitality data sets 120 within each cluster 160. In one or more embodiments, graphical charts may be used to determine outliers, similarities and the like.

With continued reference to FIG. 1, apparatus 100 may further include a display device communicatively connected to at least a processor 108. “Display device” for the purposes of this disclosure is a device configured to show visual information. In some cases, display device may include a liquid crystal display (LCD), a cathode ray tube (CRT), a plasma display, a light emitting diode (LED) display, and any combinations thereof. Display device may include, but is not limited to, a smartphone, tablet, laptop, monitor, tablet, and the like. Display device may include a separate device that includes a transparent screen configured to display computer generated images and/or information. In some cases, display device may be configured to visually present one or more data through GUI 168 to a user, wherein a user may interact with the data through GUI 168. In some cases, a user may view GUI 168 through display.

With continued reference to FIG. 1, providing clusters 160 to cluster analysis platform 164 may allow for training of a prediction machine learning model 172. “Prediction machine learning model” for the purposes of this disclosure is a machine learning model configured to receive health records, such as vitality data set 120 or augmented data set 156 and output predicted outcomes. For example, and without limitation, prediction machine learning model 172 may determine that an individual, as indicated by vitality data set 120, may be more prone to cancer, may require surgery in the near future costing a particular amount, may require medical attention soon, may require medical intervention to prevent a particular medical condition and the like. In one or more embodiments, clusters 160 may indicate that individuals with a particular set of traits may be more prone to a particular medical condition. In one or more embodiments, prediction machine learning model 172 may be used to generate or determine predictive outputs. A “Predictive output” for the purposes of this disclosure is projected outcome associated with an individual's health based on a received input. For example, and without limitation information associated with a future patient may be used to predict future medical conditions and associated costs. In one or more embodiments, predictive outputs may include but are not limited to, predicted diseases states for an individual in the future, predicted health outcomes based on a medication taken, predicted, health increases or health decreases, predicted health outcomes if one or more particular medications are taken. Predictive health outcomes based on a persons financial status, predicted further treatment events 140 and the like. In one or more embodiments, identification of clusters 160 may allow for training of prediction machine learning model 172 wherein individuals with similar biological or physical traits as indicated within a cluster 160 may be prone to the disease mentioned within the cluster 160. For example, and without limitation, a cluster 160 showing that overweight individuals above 50 may be more prone to cancer may be used to show that a particular patient who is overweight and above 50 may be more prone to cancer. In one or more embodiments, similarities between patients may not seem apparent until auxiliary information 128, such as cost of care is added. In one or more embodiments, absent auxiliary information 128 correlations may be inaccurate. In one or more embodiments, prediction training data 176 may include a plurality of vitality data sets 120 and/or augmented data sets 156 correlated to a plurality of predictive outputs, wherein predictive outputs may include predicted outcomes of the patient. In one or more embodiments, cluster analysis platform 164 may generate and/or update prediction training data 176 by generating data points for each element within augmented data set 156 and associated levels, wherein clusters 160 may be used as labels. For example, and without limitation, features may include age, physical health, medical background and the like and the labels may include clusters 160 such as a particular medication condition. In an embodiment, prediction machine learning model 172 may be iteratively trained wherein inputs such as vitality data set 120 may indicate a correlated output such as a possible future disease, a possible future cost and the like. In one or more embodiments, prediction machine learning model 172 may include a regression model wherein values are predicted based on inputs. In one or more embodiments, augmented data set 156 may be converted into training data wherein features such as an age, medical history and the like may be given a value such that prediction machine learning model 172 may predict numerical outcomes. In one or more embodiments, the numerical outcomes may be used to determine the health of the patient. In one or more embodiments, prediction machine learning model 172 may include a clustering model wherein patients that fit within a particular cluster 160 may be labeled similarly. In one or more embodiments, cluster analysis platform 164 may be configured to generate prediction training data 176 and iteratively train prediction machine learning model 172, wherein prediction machine learning model 172 may be trained following each iteration. In one or more embodiments, prediction machine learning model 172 may be configured to identify patterns within clusters 160 wherein patterns may be used to predict outcomes of future patients. For example, and without limitation, prediction machine learning model 172 may identify a pattern such as weight gain with an association to a worse physical state. Similarly, prediction machine learning model 172 may identify a pattern such as high costs of medication with an association to worse medical conditions due to a lack on financial health of the patients.

With continued reference to FIG. 1, in one or more embodiments, providing at least one cluster 160 to cluster analysis platform 164 may include generating prediction training data 176 including a plurality of augmented data sets 156 correlated to a plurality of predictive outputs, iteratively training a prediction machine learning model as a function of the prediction training data and the similarity datum 180 and determining predictive outputs using the prediction machine learning model. In one or more embodiments, similarity datum 180 may be used to iteratively train prediction machine learning model wherein similarity datum 180 may include inputs containing similarities and outputs containing disease states and/or clusters 160. In one or more embodiments, similarity datum 180 may be used to correlate inputs and outputs wherein individuals with similar physical traits may be used to determine which cluster a person may belong to. For example, and without limitation, a cluster having similarities such as high blood pressure and high cholesterol may be used to indicate that an individual having high blood pressure and high cholesterol may belong in a particular cluster 160. In one or more embodiments, clusters may be grouped based on medications taken, diseases states and the like wherein assigning an individual to a particular cluster may indicate that they are more prone to a particular diseases and the like. In one or more embodiments, similarity datum 180 may be used to predict outcomes wherein individuals having similar characteristics may be predicted to eventually contain a particular disease. In one or more embodiments, similarity datum 180 may be used to train prediction machine learning model wherein similarity datum 180 may be used to correlate inputs (e.g. the elements identified to be similarities) to predictive outputs (e.g. the cluster that the user may eventually be assigned to). In one or more embodiments, similarity datum 180 may be used to iteratively train prediction machine learning model 172 wherein prediction machine learning model 172 may contain updated correlated inputs and outputs. In one or more embodiments, cluster analysis platform 164 may be used to train prediction machine learning model 172. In one or more embodiments, prediction machine learning model may be configured to receive vitality data sets 120 and/or electronic health records and outputs predictive outputs.

With continued reference to FIG. 1, in one or more embodiments, cluster analysis platform 164 may utilize transfer learning in order to train prediction machine learning model 72. In one or more embodiments, transfer learning may include a process in which an existing machine learning model is trained on new problems. In one or more embodiments, transfer learning with respect to prediction machine learning model may include new processes to identify diseases states, new processes to determine predictive outputs and the like. In one or more embodiments, similarity datum 180 may be used for transfer learning wherein prediction machine learning model may be trained on new correlated inputs and outputs. In one or more embodiments, similarity datum 180 may contain correlated inputs and outputs wherein the correlated inputs include elements with a high degree fo similarity and outputs may include the labeling of the cluster 160 to which the data was grouped to. In one or more embodiments, similarity datum 180 may be used for transfer learning wherein prediction machine learning model may be trained on a narrower data set to capture various outcomes. In one or more embodiments, similarity datum 180 may be used to train pre-trained machine learning models. In one or more embodiments, pretrained machine learning model may include broad correlated inputs and outputs wherein similarity datum 180 may be used to capture more narrow areas in order to generate more accurate predictive outputs. In one or more embodiments, machine learning models may be pre-trained with data wherein similarity datum 180 may be used to add on to the machine learning models to create more accurate outputs.

With continued reference to FIG. 1, cluster analysis platform 164 may be used to identify patterns within each cluster 160. In one or more embodiments, cluster analysis platform 164 may utilize one or more machine learning models and/or neural networks as described in this disclosure to derive patterns within each cluster 160 that may be associated with a particular health and/or medical condition. In one or more embodiments, cluster analysis platform 164 may be configured to generate pattern data wherein pattern data may indicate various patterns and/or similarities within each cluster 160. In one or more embodiments, pattern data may be used to identify the root of a medical issue. In one or more embodiments, pattern data may be used to identify future medical issues due to similarly detected patterns. In one or more embodiments, cluster analysis platform 164 may be configured to determine why a first cluster 160 contained a first disease and why a second cluster 160 did not contain the same disease. In one or more embodiments, pattern recognition may include the user of supervised learning, unsupervised learning, deep learning, nearest neighbor methods and the like. In one or more embodiments, cluster analysis platform 164 may be configured to utilize a machine learning model such as any machine learning model as described in this disclosure to compare clusters 160 and determine patterns between clusters 160. In one or more embodiments, the machine learning model may be configured to receive a plurality of clusters 160 and determine patterns within and/or between clusters 160. For example, and without limitation, cluster analysis platform 164 may determine that a particular group of patients may have had more successful outcomes in their medical treatment due to the cost of drug prices in comparison to another group of patients who had less successful outcomes. In one or more embodiments, clusters 160 may be identified based on cost of care wherein patients with higher costs of care may be associated with more suitable outcomes than those associated with lower costs of care. In an embodiments, supplementing vitality data sets 120 with auxiliary information 128 allows for cluster analysis platform 164 to consider additional variables that may contribute to an undesired and/or desired outcome. In one or more embodiments, cluster analysis platform 164 may be used to generate pattern data. In one or more embodiments, pattern data may include correlations between vitality data sets 120 due to similar and/or separate quantitative data points 132. In one or more embodiments, pattern data may include information that may have contributed to a particular medical result, such as surgery, a misdiagnoses, and the like.

With continued reference to FIG. 1, cluster analysis platform 164 may be configured to receive an electronic health record such as one pertaining to a patient and output prediction data wherein prediction data may include predicted costs of medication, predicted medical treatments and the like. In one or more embodiments, cluster analysis platform 164 may provide information indicating treatments, medication and the like that may aid a patient in the prevention of various predicted diseases and/or medical costs. In one or more embodiments, processor 108 may be configured to receive an electronic health record, such as vitality data set, update cluster analysis platform 164 by providing clusters 160 to cluster analysis platform 164 as described above and generate prediction data.

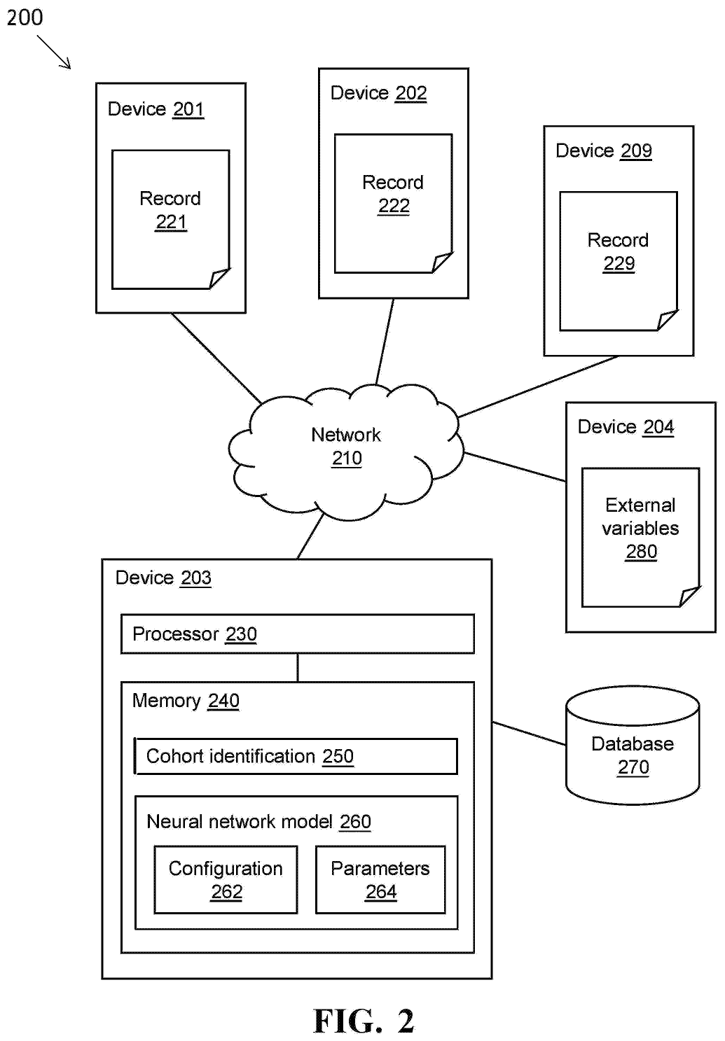

FIG. 2 is a simplified diagram of a system 200 for identifying clinical cohorts according to some embodiments. System 200 includes a plurality of devices 201-209 that are communicatively coupled via a network 210. Devices 201-209 generally include computer devices or systems, such as personal computers, mobile devices, servers, or the like. Network 1210 can include one or more local area networks (LANs), wide area networks (WANs), wired networks, wireless networks, the Internet, or the like. Illustratively, devices 201-209 may communicate over network 210 using the TCP/IP protocol or other suitable networking protocols.

One or more of devices 201-209 can store electronic health records 221-229 and/or access electronic health records 222-229 via network 220. For example, as depicted in FIG. 2, devices 201, 202, and 209 store electronic health records 221, 222, and 229, respectively, and device 203 accesses electronic health records 221-229 via network 220. Electronic health records 221-229 can include a variety of digitized patient healthcare information such as patient data (e.g., name, age, gender, demographics, medical history, etc.), physician's notes, measurement and test results, imaging results, diagnoses, prescriptions, and the like.

Illustratively, electronic health records 221-229 can be formatted as text documents, images, videos, database files, other types of digital files (e.g., raw data from medical instruments), or any other suitable format. Electronic health records 221-229 can be heterogeneous (e.g., of different formats or file types) or homogenous (e.g., of the same format or file type), and can include structured or unstructured data. In some embodiments, such as when devices 201-209 are associated with different institutions (e.g., different health care providers), the types and formats of data in electronic health records 221, 221, and 229 may vary across institutions. For efficient storage and/or transmission via network 210, records 221-229 may be compressed prior to or during transmission via network 210. Security measures such as encryption, authentication (including multi-factor authentication), SSL, HTTPS, and other security techniques may also be applied.

According to some embodiments, device 203 may access one or more electronic health records 222-229 by downloading electronic health records 221-229 from devices 201, 202, and 209. Moreover, one or more of devices 201, 202, or 209 can upload or export electronic health records 222-229 to device 203. Electronic health records 221-229 may be updated at various times. Accordingly, device 203 may access electronic health records 221-229 multiple times at various intervals (e.g., periodically) to obtain up-to-date records.

In some embodiments, when device 203 accesses data from devices 201, 202, and 209, device 203 may be restricted to access de-identified electronic health records 221-229 from which personally identifiable information has been removed. In this manner, devices 201, 202, and 209 may serve as repositories for de-identified electronic health records. This facilitates the ability of device 203 to analyze data and gain insights from healthcare data generated by multiple sources (e.g., multiple different healthcare institutions associated with devices 201, 202, and 209) while complying with applicable privacy regulations.

As depicted in FIG. 2, device 203 includes a processor 230 (e.g., one or more hardware processors) coupled to a memory 240 (e.g., one or more non-transitory memories). Memory 240 stores instructions and/or data corresponding to a clinical cohort identification program 250

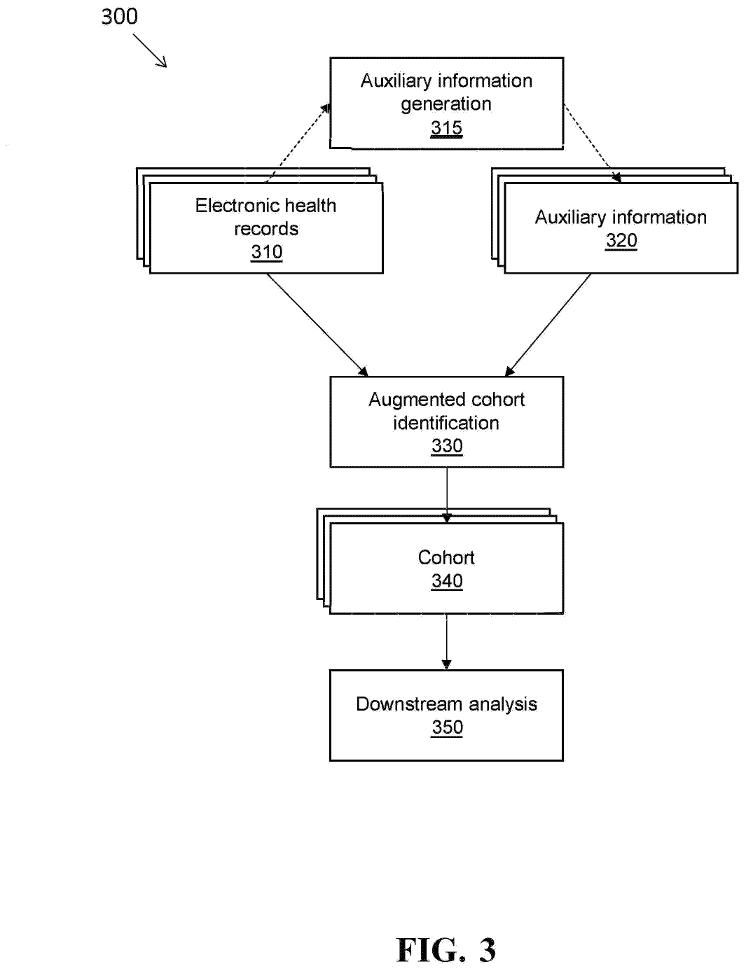

When executed by processor 230, clinical cohort identification program 250 causes processor 230 to perform operations associated with identifying clinical cohorts based on electronic health records 221-229. Illustrative embodiments of data flows implemented by clinical cohort identification program 250 are described in further detail below with reference to FIG. 3.

During or after execution of clinical cohort identification program 250, processor 230 may execute one or more neural network models, such as neural network model 260. Neural network model 260 is trained to make predictions (e.g., inferences) based on input data. Neural network model 260 includes configuration 262, which defines a plurality of layers of neural network model 260 and the relationships among the layers. Illustrative examples of layers include input layers, output layers, convolutional layers, densely connected layers, merge layers, and the like. In some embodiments, neural network model 260 may be configured as a deep neural network with at least one hidden layer between the input and output layers. Connections between layers can include feed-forward connections or recurrent connections.

Device 203 may be communicatively coupled to a database 270 or another suitable repository of digital information. For example, database 270 may be configured as a structured database with contents organized according to a schema or other logical relationships (e.g., relational database). In some embodiments database 270 may be configured as a non-relational database, a semi-structured database, an unstructured database, a key-value store, or the like. Although database 270 is depicted as being coupled directly to device 203, it is to be understood that a variety of other arrangements are possible. For example, database 270 may be stored in memory 240, accessed via network 210 or the like.

As discussed above, electronic health records 221-229 may provide an incomplete picture of the care provided to a patient. These gaps, in turn, may limit the performance of clinical cohort identification program 250, for example, by constraining the types of cohorts that can be identified or reducing the efficiency with which they are identified. Moreover, the gaps may limit the efficiency of neural network model 260 by decreasing the amount, accuracy, and/or relevance of the input data. For example, as a workaround to the gaps in electronic health records 221-229, clinical cohort identification program 250 may be programmed to identify clinical cohorts that are smaller or less relevant to the tasks performed by neural network model 260. When neural network model 260 receives a small or marginally relevant cohort as an input or as training data, this may reduce the accuracy of network model 260 and/or increase its training time.

Therefore, according to some embodiments, device 203 may be configured to access one or more external variables 280 that may, at least in part, fill the information gaps in electronic health records 221-229. Clinical cohort identification program 250 may incorporate external variables 280 into its process of identifying clinical cohorts, resulting in a more complete picture of the care provided to each patient. For example, as described in further detail below, external variables 280 may supply information associated with a patient's cost of care which is missing from electronic health records 221-229. Cost information allows clinical cohort identification program 250 to identify cohorts based on cost of care (e.g., stratification of patients with higher or lower costs of care).

As shown in FIG. 1, the external variables 280 are accessed from device 204. In some embodiments, device 204 may be physically and/or administratively separate from devices 201, 202, and 209 from which electronic health records 221-229 are accessed. For example, device 204 may be associated with a different institution from devices 201, 202, and 209, in which case device 204 may be located on different premises and may be subject to distinct requirements for securely accessing external variables 280. In embodiments where external variables 280 correspond to cost information, device 204 may be associated with an insurance entity or a government agency that serves as a repository for cost information whereas devices 201, 202, and 209 may be associated with one or more medical institutions that treat the patients.

FIG. 3 is a simplified diagram of a data flow 300 for identifying clinical cohorts according to some embodiments. In some embodiments consistent with FIG. 1, data flow 300 may be implemented using various components and/or features of system 200, as further described below.

As depicted in FIG. 3, a set of electronic health records 310 and auxiliary information 320 are provided to an augmented cohort identification module 330. Based on electronic health records 310 and auxiliary information 320, augmented cohort identification module 330 identifies one or more clinical cohorts 340, which are provided to a downstream analysis module 250 for further analysis and/or processing.

In embodiments consistent with FIG. 2, electronic health records 310 may generally correspond to electronic health records 221-229 (which may be de-identified), and auxiliary information 320 may generally correspond to external variables 280. Consequently, as described above for FIG. 2, auxiliary information 320 may supply missing, augmented, complementary, or otherwise additional information that augmented cohort identification module 330 can use in conjunction with electronic health records 310 to identify clinical cohorts 340.

In some embodiments, auxiliary information 320 may be generated using an auxiliary information generation module 315. Auxiliary information generation module 315 may generate auxiliary information 320 based on electronic health records 221-229, external variables separate from electronic health records 221-229 (e.g., external variables 280), or a combination thereof. Illustrative examples of processes for generating auxiliary information 320 are described in further detail below with reference to FIGS. 5-10. Although auxiliary information generation module 315 is depicted in FIG. 3 as a separate module from augmented cohort identification module 330 for illustrative purposes, it is to be understood that other arrangements are possible. For example, auxiliary information generation module 315 may be within or may carry out processes in conjunction with augmented cohort identification module 330.

In some embodiments, auxiliary information 320 may include the cost of care to a payer, hereinafter referred to as “cost of care” information. Cost of care may include virtually any form of cost for care, including but not limited to a cost for a patient encounter a drug cost, a procedure cost, or the like. Consistent with such embodiments, electronic health records 310 and auxiliary information 320 may be provided to augmented cohort identification module 330 from different entities, e.g., a healthcare provider that maintains patient records and an insurance provider or government agency that maintains cost of care data, respectively.

To identify clinical cohorts, augmented cohort identification module 330 may be configured to merge electronic health records 310 and auxiliary information 320. For example, when auxiliary information 320 includes cost of care data, augmented cohort identification module 330 may be configured to identify references to the administration of such care within electronic health records 310. Such references may be in structured text form, in unstructured text form, embedded on images, accompanying images as metadata, or the like. By merging electronic health records 310 and auxiliary information 320, augmented cohort identification module 330 can benefit from the use of auxiliary information 320 when identifying clinical cohorts among electronic health records 310.

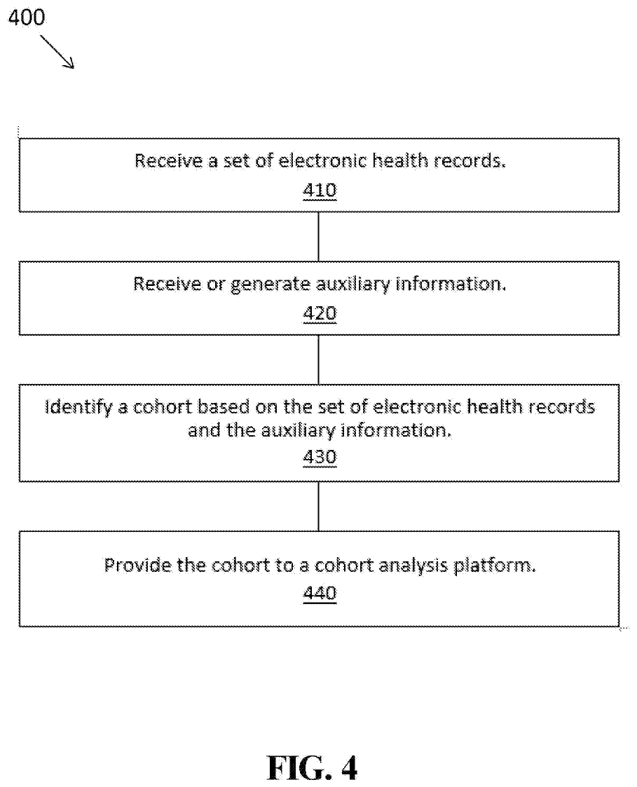

FIG. 4 is a simplified diagram of method 400 for identifying clinical cohorts according to some embodiments. According to some embodiments consistent with FIGS. 2-3, method 400 may be performed by processor 130 during the execution of clinical cohort identification program 150. For example, method 400 may be performed using augmented clinical cohort identification module 230.

At step 410, a set of electronic health records (e.g., electronic health records 310) is received. In some embodiments, the electronic health records may be received from one or more healthcare providers, such as a hospital, clinic, doctor's office, or the like. As described above, electronic health records may include various types of patient data in various formats and/or data structures. As further described above, the electronic health records may be deidentified and may be collected from within and across medical institutions and/or facilities that are the owners of the de-identified data.

At step 420, auxiliary information (e.g., auxiliary information 320) is received or generated (or both). For example, the auxiliary information may include cost of care information, as described above and/or other types of information which are not typically included in electronic health records, but which may be useful for identifying clinical cohorts. In some embodiments, at least a portion of the auxiliary information may be received from an external source that is separate from the source of the electronic health records (e.g., the sources may be associated with different entities or institutions). In some embodiments, the auxiliary information may be generated based at list in part on the electronic health records received at step 410. Illustrative examples of processes for generating auxiliary information 320 are described in further detail below with reference to FIGS. 5-10.

At step 430, one or more clinical cohorts (e.g., clinical cohorts 240) are identified based on the set of electronic health records and the auxiliary information. In some embodiments, the clinical cohort may be identified by merging the auxiliary information with the set of electronic health records. For example, when auxiliary information includes cost of care data, the auxiliary information may be merged with the set of electronic health records by identifying references to the administration of care for which cost of care data is available within the electronic health records. The merged data may then be used to identify cohorts based on more complete patient information than would be achievable with electronic health records alone. For example, when auxiliary information includes cost of care data, clinical cohorts may stratify based on average cost of care (e.g., high, medium, and low cost cohorts), enabling new insights and more efficient downstream analysis of the cohorts.

At process step 440, the one or more clinical cohorts are provided to a cohort analysis platform. For example, the cohort analysis platform may provide an interface for a clinician (or other user) to analyze the one or more cohorts and derive insights. In some embodiments, the cohort analysis platform may be configured to train a neural network model to make predictions using the one or more clinical cohorts as training and/or test data. Consistent with such embodiments, the ability of method 400 to provide complete and accurate clinical cohorts that are formed using auxiliary information in conjunction with electronic health records may improve the performance of the trained neural network model. For example, in some embodiments, the neural network model may be trained to perform tasks that would not otherwise be achievable using electronic health records without auxiliary information.

FIG. 5 is a simplified diagram of a method 500 for generating cost of care data and merging cost of care data with electronic health records according to some embodiments.

According to some embodiments consistent with FIGS. 1-4, method 500 may be performed by processor 230 during the execution of clinical cohort identification program 150. For example, method 500 may be performed using augmented clinical cohort identification module 230.

At process step 510, a patient event is identified based on a set of electronic health records (e.g., electronic health records 310). The patient event may be identified from electronic health records based on a variety of information in the records such as structured text, unstructured text, images, metadata, and the like. In some examples, the patient event may include a type of patient event that is likely to result in costs to the payer, such as an inpatient event, an outpatient event, a medication event, or the like. For example, when a patient enters a hospital for a surgical procedure, the patient is likely to incur costs as part of the inpatient event.

Similarly, when a patient fills a prescription, the patient is likely to incur costs as part of the medication event. However, the costs associated with these events are not typically included in the patient's electronic health records, even though it may be desirable to form clinical cohorts that take into account such costs. Indeed, the costs may not be set at the time of the patient event, as the process of determining costs may be separate from the event, e.g., based on input from an insurance company or government agency.

At process step 520, one or more codes associated with the patient event is identified. The codes may correspond to structured or standardized information that is used to characterize the patient event, or a portion thereof, typically for billing purposes. For example, inpatient or outpatient events may be associated with a diagnosis code (e.g., an International Classification of Diseases (ICD) code), procedure code (e.g., a Current Procedural Terminology (CPT) code, an Inpatient Prospective Payment System (IPPS) code, or an Outpatient Prospective Payment System (OPPS) code), a diagnosis-related group (DRG) code, an Ambulatory Payment Classifications (APC) code, or the like. Medications may be associated with a code such as National Drug Code (NDC). Illustrative examples of codes associated with patient events and processes for identifying such codes are identified in FIGS. 5-8. Such codes may be contained explicitly in the electronic health records or may be derived based on other information in the electronic health records.

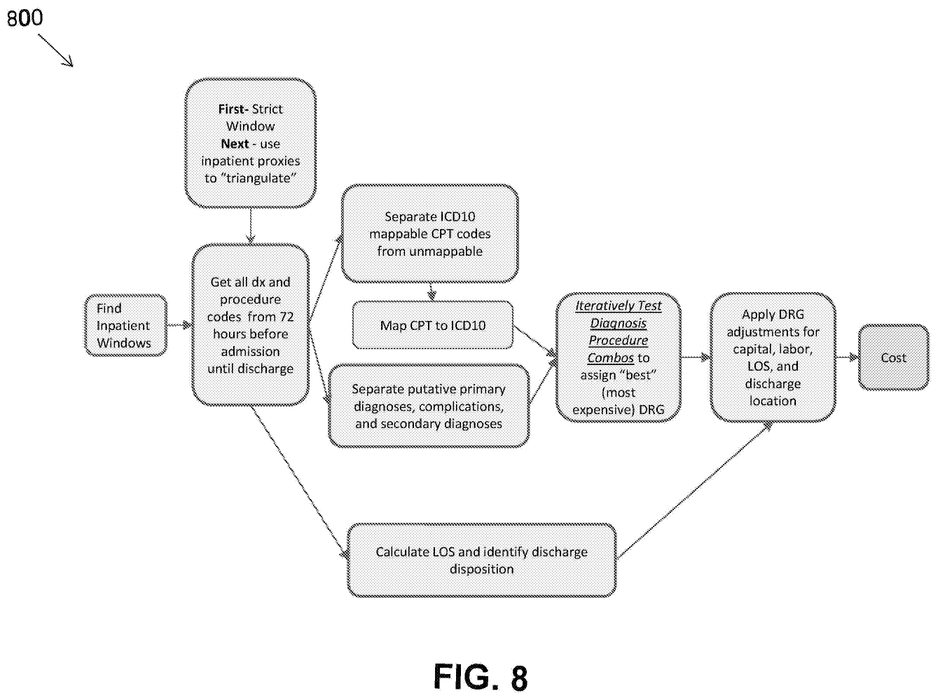

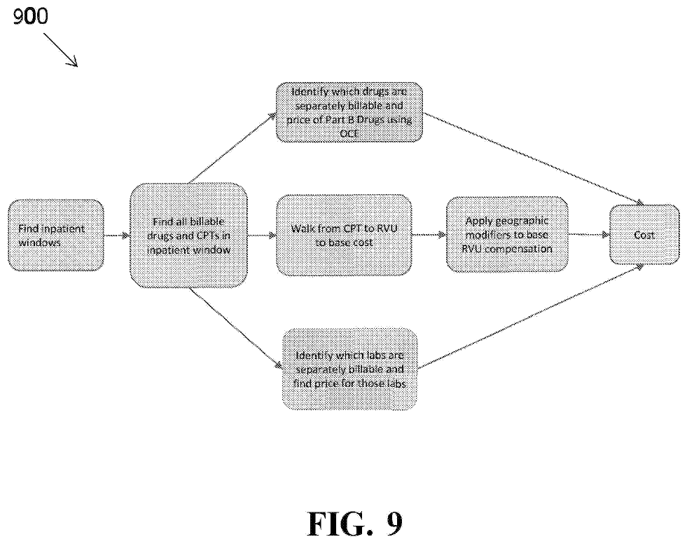

In some embodiments, the one or more codes may be identified by determining a time window associated with a patient event and identifying one or more codes that occur within the time window. For example, as shown in the example of FIG. 8-9, the time window for an inpatient event is set to begin 72 hours before admission and to end at discharge.

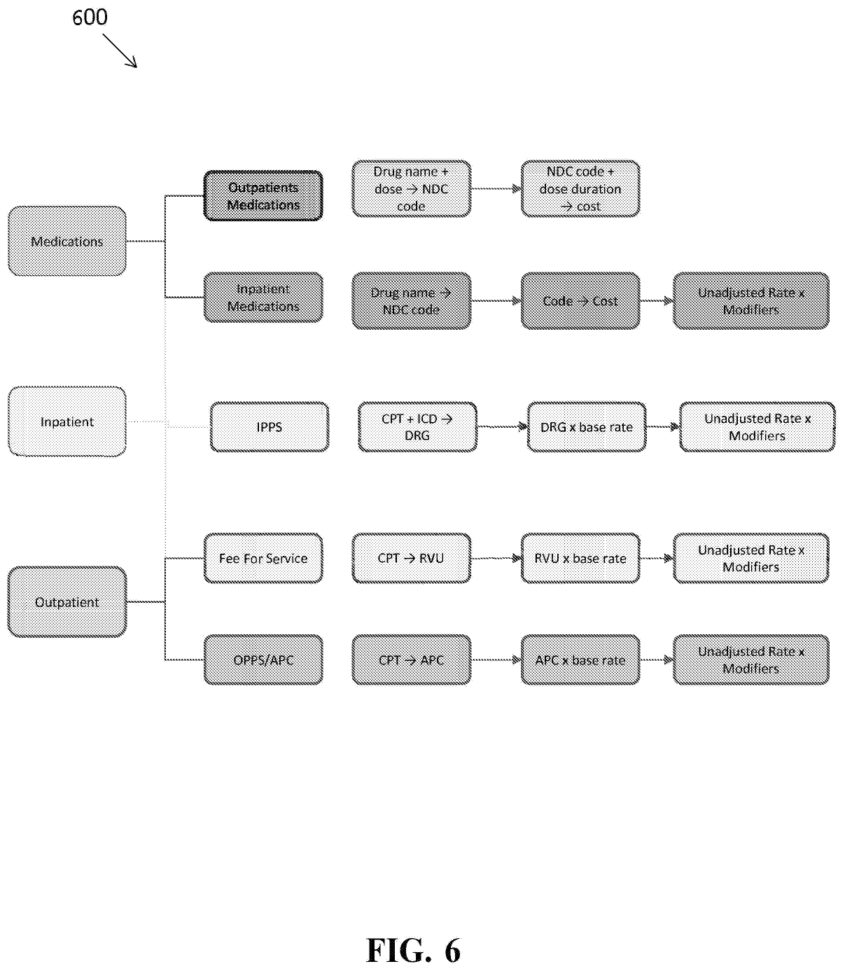

At process step 530, a cost associated with the patient event is determined based on the one or more codes. For example, the one or more codes may be used to estimate a Centers for Medicare & Medicaid Services (CMS) reimbursement associated with the patient event, which may be used to determine the estimated cost of the patient event. In some embodiments, the one or more codes may be converted to an unadjusted rate, which may be adjusted based on one or more modifiers to determine the cost. For example, as shown in FIGS. 6, 8, and 9, the modifiers may result in adjustments to account for capital, labor, length of stay (LOS), discharge location, and geographic modifiers. In some embodiments, where the set of codes may result in different possible reimbursement amounts or estimated costs, the combination of codes that results in the highest cost may be selected.

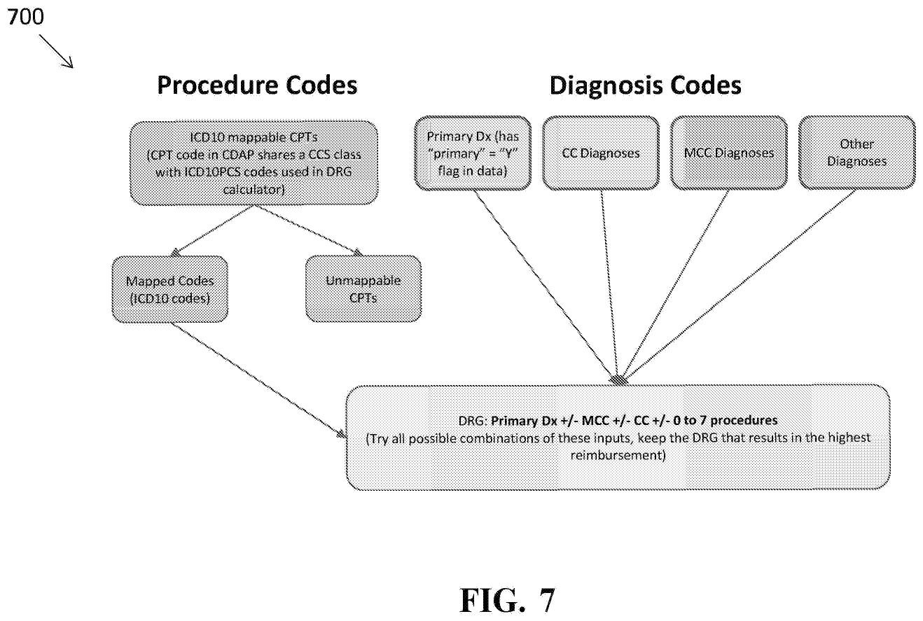

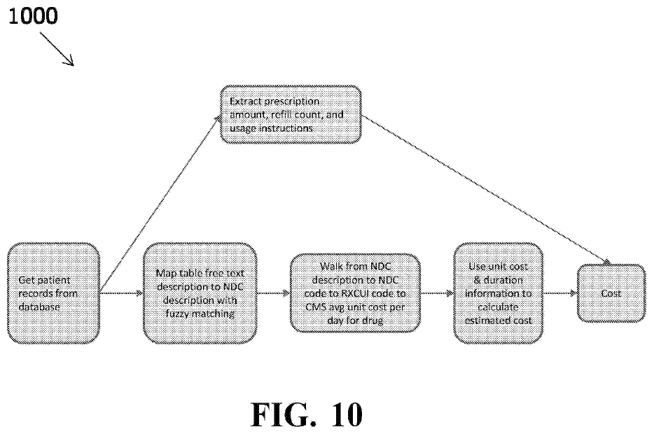

FIGS. 6-10 are simplified diagrams of methods 600-1000 for generating cost of care data and merging cost of care data with electronic health records according to some embodiments. FIGS. 6-10 generally depict further, non-limiting details associated with method 500. For example, FIG. 6 depicts illustrative processes for determining certain types of costs for medication events, inpatient events, and outpatient events (e.g., at process steps 520-530). FIGS. 7-8 depict illustrative processes for determining a diagnosis-related group (DRG) code based on procedure and diagnosis codes for an inpatient event, where the combination of procedure and diagnosis codes that results in the highest reimbursement is selected (e.g., at process steps 520-530). FIG. 9 depicts an illustrative process for determining physician services costs for an inpatient event (e.g., at process steps 520-530). FIG. 10 depicts a process for determining costs associated with a medication event (e.g., prescription drugs), e.g., at process steps 510-530.

The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of nonvolatile memory, including by way of example semiconductor memory devices, (e.g., EPROM EEPROM and flash memory devices). magnetic disks, (e.g., internal hard disks or removable disks); magneto optical disks• and optical disks (e.g., CD and DVD disks). The processor and the memory can be supplemented by, or incorporated in, special purpose logic circuitry.

To provide for interaction with a user, the subject matter described herein can be implemented on a computer having a display device, e.g., a CRT (cathode ray tube) or LCD (liquid crystal display) monitor, for displaying information to the user and a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back end component (e.g., a data server), a middleware component (e.g., an application server), or a front end component (e.g., a client computer having a graphical user interface or a web browser through which a user can interact with an implementation of the subject matter described herein), or any combination of such back end, middleware, and front end components. The components of the system can be interconnected by any form or medium of digital data communication, e.g., a communication network. Examples of communication networks include a local area network (“LAN”) and a wide area network (“WAN”), e.g., the Internet.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. It is important, therefore, that the claims be regarded as including such equivalent constructions insofar as they do not depart from the spirit and scope of the disclosed subject matter. Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter, which is limited only by the claims which follow.

Referring now to FIG. 11, an exemplary embodiment of a machine-learning module 1100 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 1104 to generate an algorithm instantiated in hardware or software logic, data structures, and/or functions that will be performed by a computing device/module to produce outputs 1108 given data provided as inputs 1112; this is in contrast to a non-machine learning software program where the commands to be executed are determined in advance by a user and written in a programming language.

Still referring to FIG. 11, “training data,” as used herein, is data containing correlations that a machine-learning process may use to model relationships between two or more categories of data elements. For instance, and without limitation, training data 1104 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 1104 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1104 according to various correlations; correlations may indicate causative and/or predictive links between categories of data elements, which may be modeled as relationships such as mathematical relationships by machine-learning processes as described in further detail below. Training data 1104 may be formatted and/or organized by categories of data elements, for instance by associating data elements with one or more descriptors corresponding to categories of data elements. As a non-limiting example, training data 1104 may include data entered in standardized forms by persons or processes, such that entry of a given data element in a given field in a form may be mapped to one or more descriptors of categories. Elements in training data 1104 may be linked to descriptors of categories by tags, tokens, or other data elements; for instance, and without limitation, training data 1104 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 11, training data 1104 may include one or more elements that are not categorized; that is, training data 1104 may not be formatted or contain descriptors for some elements of data. Machine-learning algorithms and/or other processes may sort training data 1104 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1104 to be made applicable for two or more distinct machine-learning algorithms as described in further detail below. Training data 1104 used by machine-learning module 1100 may correlate any input data as described in this disclosure to any output data as described in this disclosure. As a non-limiting illustrative example inputs may include inputs such as vitality data set and outputs may include outputs such as auxiliary information.

Further referring to FIG. 11, training data may be filtered, sorted, and/or selected using one or more supervised and/or unsupervised machine-learning processes and/or models as described in further detail below; such models may include without limitation a training data classifier 1116. Training data classifier 1116 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 1100 may generate a classifier using a classification algorithm, defined as a processes whereby a computing device and/or any module and/or component operating thereon derives a classifier from training data 1104. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1116 may classify elements of training data to [something that characterizes a sub-population, such as a cohort of persons and/or other analyzed items and/or phenomena for which a subset of training data may be selected].

Still referring to FIG. 11, computing device 1104 may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 1104 may then calculate a likelihood table by calculating probabilities of different data entries and classification labels. Computing device 1104 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 11, computing device 1104 may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 11, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where αᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 11, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 11, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 11, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, santization may include utilizing algorithms for identifying duplicate entries or spell-check algorithms.

As a non-limiting example, and with further reference to FIG. 11, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 11, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 11, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 11, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 11, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 11, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 11, machine-learning module 1100 may be configured to perform a lazy-learning process 1120 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1104. Heuristic may include selecting some number of highest-ranking associations and/or training data 1104 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 11, machine-learning processes as described in this disclosure may be used to generate machine-learning models 1124. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 1124 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 1124 may be generated by creating an artificial neural network, such as a convolutional neural network comprising an input layer of nodes, one or more intermediate layers, and an output layer of nodes. Connections between nodes may be created via the process of “training” the network, in which elements from a training data 1104 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 11, machine-learning algorithms may include at least a supervised machine-learning process 1128. At least a supervised machine-learning process 1128, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include inputs such as vitality data set as described above as inputs, outputs such as auxiliary information as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 1104. Persons skilled in the art, upon reviewing the entirety of this disclosure, will be aware of various possible variations of at least a supervised machine-learning process 1128 that may be used to determine relation between inputs and outputs. Supervised machine-learning processes may include classification algorithms as defined above.

With further reference to FIG. 11, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 11, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 11, machine learning processes may include at least an unsupervised machine-learning processes 1132. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 1132 may not require a response variable; unsupervised processes 1132 may be used to find interesting patterns and/or inferences between variables, to determine a degree of correlation between two or more variables, or the like.

Still referring to FIG. 11, machine-learning module 1100 may be designed and configured to create a machine-learning model 1124 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 11, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient trec boosting, and/or voting classifier methods. Machine-learning algorithms may include neural net algorithms, including convolutional neural net processes.

Still referring to FIG. 11, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 11, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 11, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 11, one or more processes or algorithms described above may be performed by at least a dedicated hardware unit 1136. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 1136 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 1136 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 1136 to perform one or more operations described herein, such as evaluation of model and/or algorithm outputs, one-time or iterative updates to parameters, coefficients, weights, and/or biases, and/or any other operations such as vector and/or matrix operations as described in this disclosure.

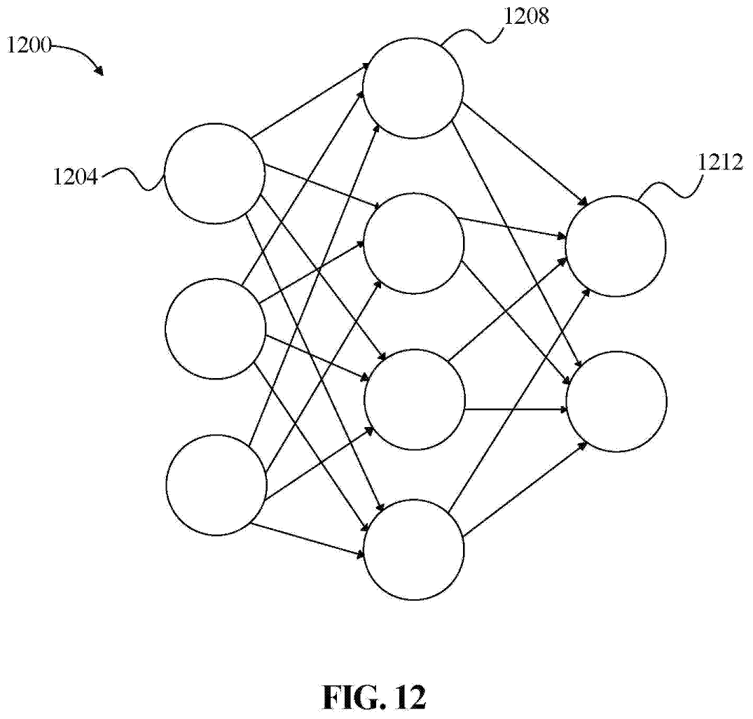

Referring now to FIG. 12, an exemplary embodiment of neural network 1200 is illustrated. A neural network 1200 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 1204, one or more intermediate layers 1208, and an output layer of nodes 1212. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

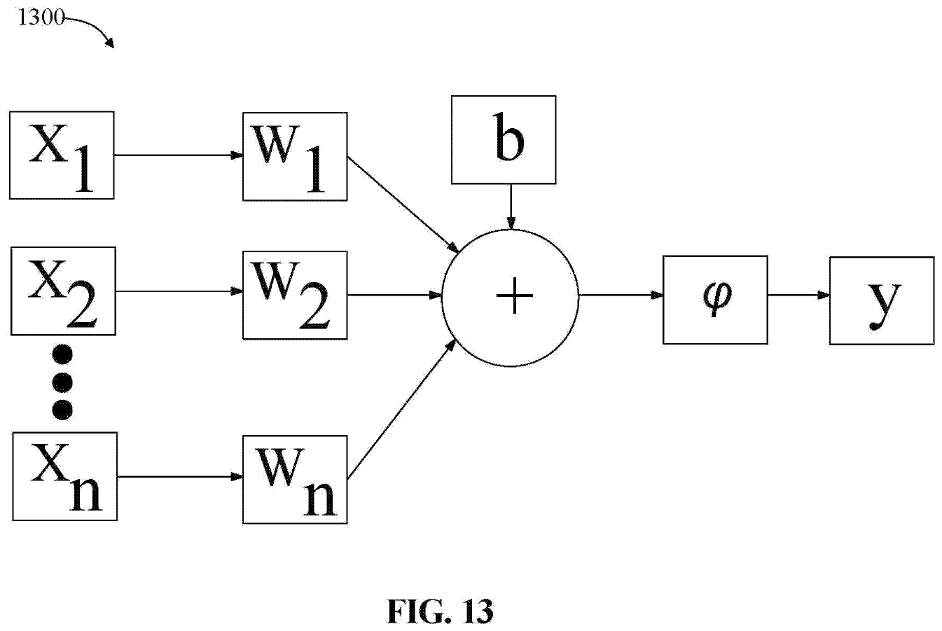

Referring now to FIG. 13, an exemplary embodiment of a node 1300 of a neural network is illustrated. A node may include, without limitation a plurality of inputs xi that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given
input x, a tanh (hyperbolic tanget) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f (x)=max(αx,x) for some α, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as function such as f(x)=x*sigmoid(x), a Gaussian error linear unit function such as f(x)=α(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs x; that may be used as activation functions. As a non-limiting and illustrative example, node may perform a weighted sum of inputs using weights w; that are multiplied by respective inputs xi. Additionally or alternatively, a bias b may be added to the weighted sum of the inputs such that an offset is added to each unit in the neural network layer that is independent of the input to the layer. The weighted sum may then be input into a function q, which may generate one or more outputs y. Weight w; applied to an input x; may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

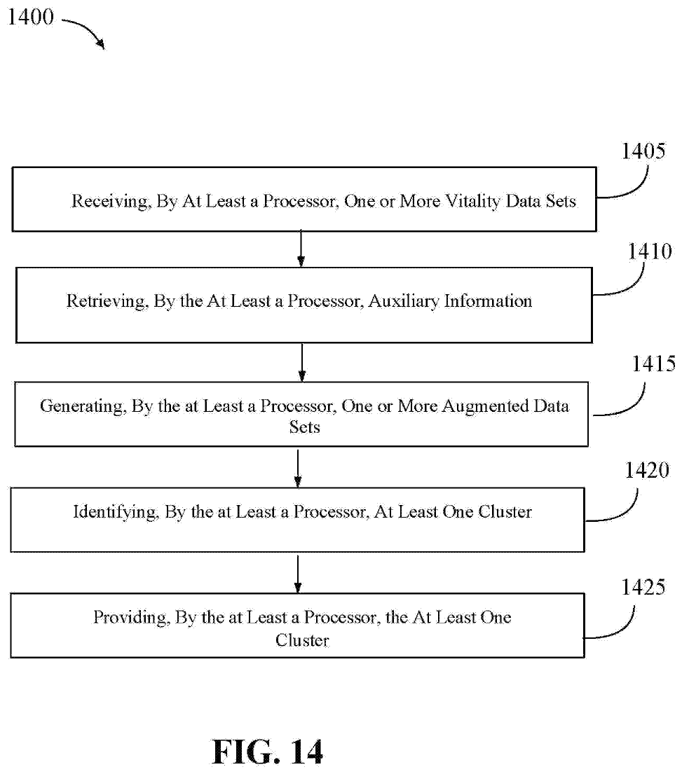

Referring now to FIG. 14, an example method 1400 for identifying clusters based on augmented data sets is described. At step 1405, method 1400 includes receiving, by at least a processor, one or more vitality data sets. In one or more embodiments, the one or more vitality data sets includes at least a data file. This may be implemented with reference to FIGS. 1-14 and without limitation.

With continued reference to FIG. 14, at step 1410 method 1400 includes retrieving, by at least a processor, auxiliary information for the one or more vitality data sets. In one or more embodiments, auxiliary information includes one or more quantitative data point used to quantify one or more elements with the one or more vitality data sets. In one or more embodiments, retrieving the auxiliary information includes identifying one or more treatment events with each of the one or more vitality data sets and retrieving the one or more quantitative data points associated with each of the one or more vitality data sets for each treatment event of the one or more treatment events. In one or more embodiments, identifying the one or more treatment events includes generating a timeframe for each treatment event of the one or more treatment events and retrieving the one or more quantitative data points associated with each of the one or more vitality data sets for each treatment event includes grouping the one or more quantitative data points for each treatment event as a function for the timeframe. In one or more embodiments, retrieving the auxiliary information for the one or more vitality data sets includes retrieving the auxiliary information using an auxiliary module. In one or more embodiments, the auxiliary module is configured to receive the one or more vitality data sets, determine at least one missing element within the one or more vitality data sets and retrieve the auxiliary information as a function of the missing element. In one or more embodiments, retrieving auxiliary information may include retrieving auxiliary information using a web crawler. This may be implemented with reference to FIGS. 1-14 and without limitation.

With continued reference to FIG. 14, at step 1415, method 1400 includes generating, by at least a processor, one or more augmented data sets as a function of the auxiliary information and the one or more vitality data sets. In one or more embodiments, generating the one or more augmented data sets includes identifying textual data from the at least a data file using optical character recognition. This may be implemented with reference to FIGS. 1-14 and without limitation.

With continued reference to FIG. 14, at step 1420, method 1400 includes identifying, by at least a processor, at least one cluster based on the one or more augmented data sets. In one or more embodiments, identifying the at least one cluster based on the one or more augmented data sets further includes classifying the one or more augmented data sets to the at least one cluster as a function of a classification machine learning model. This may be implemented with reference to FIGS. 1-14 and without limitation.

With continued reference to FIG. 14, at step 1425, method 1400 includes providing the at least one cluster to a cluster analysis platform, wherein the cluster analysis platform is configured to generate a similarity datum as a function of the at least one cluster and the one or more augmented data sets. In one or more embodiments, providing the at least one cluster to a cluster analysis platform includes generating prediction training data comprising a plurality of augmented data sets correlated to a plurality of predictive outputs, iteratively training a prediction machine learning model as a function of the prediction training data and the similarity datum, and determining the predictive outputs using the prediction machine learning model. This may be implemented with reference to FIGS. 1-14 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

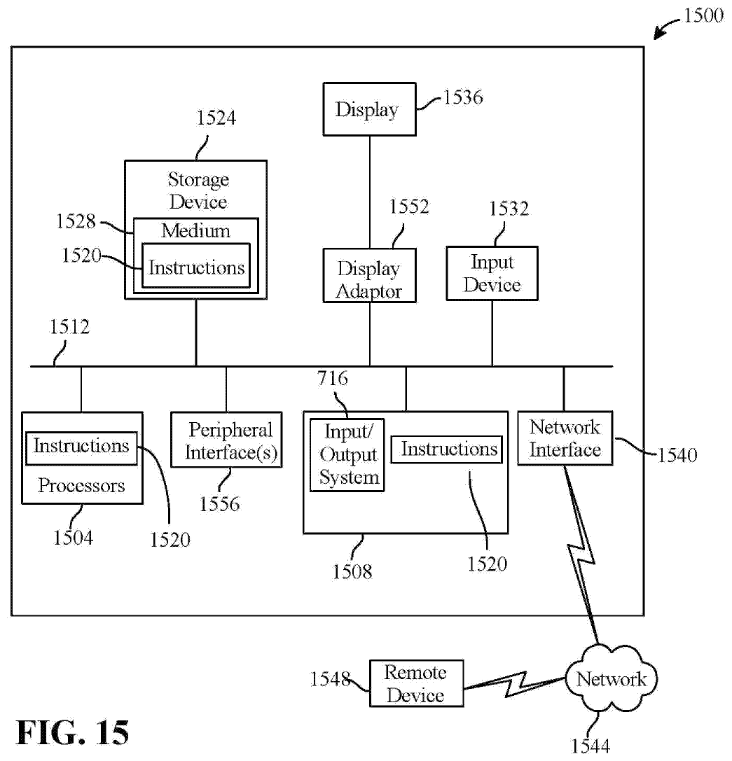

FIG. 15 shows a diagrammatic representation of one embodiment of a computing device in the exemplary form of a computer system 1500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1500 includes a processor 1504 and a memory 1508 that communicate with each other, and with other components, via a bus 1512. Bus 1512 may include any of several types of bus structures including, but not limited to, a memory bus, a memory controller, a peripheral bus, a local bus, and any combinations thereof, using any of a variety of bus architectures.

Processor 1504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1504 may be organized according to Von Neumann and/or Harvard architecture as a non-limiting example. Processor 1504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1508 may include various components (e.g., machine-readable media) including, but not limited to, a random-access memory component, a read only component, and any combinations thereof. In one example, a basic input/output system 1516 (BIOS), including basic routines that help to transfer information between elements within computer system 1500, such as during start-up, may be stored in memory 1508. Memory 1508 may also include (e.g., stored on one or more machine-readable media) instructions (e.g., software) 1520 embodying any one or more of the aspects and/or methodologies of the present disclosure. In another example, memory 1508 may further include any number of program modules including, but not limited to, an operating system, one or more application programs, other program modules, program data, and any combinations thereof.

Computer system 1500 may also include a storage device 1524. Examples of a storage device (e.g., storage device 1524) include, but are not limited to, a hard disk drive, a magnetic disk drive, an optical disc drive in combination with an optical medium, a solid-state memory device, and any combinations thereof. Storage device 1524 may be connected to bus 1512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1524 (or one or more components thereof) may be removably interfaced with computer system 1500 (e.g., via an external port connector (not shown)). Particularly, storage device 1524 and an associated machine-readable medium 1528 may provide nonvolatile and/or volatile storage of machine-readable instructions, data structures, program modules, and/or other data for computer system 1500. In one example, software 1520 may reside, completely or partially, within machine-readable medium 1528. In another example, software 1520 may reside, completely or partially, within processor 1504.

Computer system 1500 may also include an input device 1532. In one example, a user of computer system 1500 may enter commands and/or other information into computer system 1500 via input device 1532. Examples of an input device 1532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1532 may be interfaced to bus 1512 via any of a variety of interfaces (not shown) including, but not limited to, a serial interface, a parallel interface, a game port, a USB interface, a FIREWIRE interface, a direct interface to bus 1512, and any combinations thereof. Input device 1532 may include a touch screen interface that may be a part of or separate from display 1536, discussed further below. Input device 1532 may be utilized as a user selection device for selecting one or more graphical representations in a graphical interface as described above.

A user may also input commands and/or other information to computer system 1500 via storage device 1524 (e.g., a removable disk drive, a flash drive, etc.) and/or network interface device 1540. A network interface device, such as network interface device 1540, may be utilized for connecting computer system 1500 to one or more of a variety of networks, such as network 1544, and one or more remote devices 1548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1544, may employ a wired and/or a wireless mode of communication. In general, any network topology may be used. Information (e.g., data, software 1520, etc.) may be communicated to and/or from computer system 1500 via network interface device 1540.

Computer system 1500 may further include a video display adapter 1552 for communicating a displayable image to a display device, such as display device 1536. Examples of a display device include, but are not limited to, a liquid crystal display (LCD), a cathode ray tube (CRT), a plasma display, a light emitting diode (LED) display, and any combinations thereof. Display adapter 1552 and display device 1536 may be utilized in combination with processor 1504 to provide graphical representations of aspects of the present disclosure. In addition to a display device, computer system 1500 may include one or more other peripheral output devices including, but not limited to, an audio speaker, a printer, and any combinations thereof. Such peripheral output devices may be connected to bus 1512 via a peripheral interface 1556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. An apparatus for identifying clusters based on augmented data sets, the apparatus comprising:
at least a processor; and
a memory communicatively connected to the at least a processor, wherein the memory contains instructions configuring the at least a processor to:
receive one or more vitality data sets;
retrieve auxiliary information for the one or more vitality data sets;
generate one or more augmented data sets as a function of the auxiliary information and the one or more vitality data sets;
identify at least one cluster based on the one or more augmented data sets; and
provide the at least one cluster to a cluster analysis platform, wherein the cluster analysis platform is configured to generate a similarity datum as a function of the at least one cluster and the one or more augmented data sets.

2. The apparatus of claim 1, wherein:
the one or more vitality data sets comprise at least a data file; and
generating the one or more augmented data sets comprises identifying textual data from the at least a data file using optical character recognition.

3. The apparatus of claim 1, wherein the cluster analysis platform comprises a graphical user interface configured to present the similarity datum in a graphical format.

4. The apparatus of claim 1, wherein the auxiliary information comprises one or more quantitative data points used to quantify one or more elements within the one or more vitality data sets.

5. The apparatus of claim 4, wherein retrieving the auxiliary information comprises:
identifying one or more treatment events with each of the one or more vitality data sets; and
retrieving the one or more quantitative data points associated with each of the one or more vitality data sets for each treatment event of the one or more treatment events.

6. The apparatus of claim 5, wherein:
identifying the one or more treatment events comprises generating a timeframe for each treatment event of the one or more treatment events; and
retrieving the one or more quantitative data points associated with each of the one or more vitality data sets for each treatment event comprises grouping the one or more quantitative data points for each treatment event as a function for the timeframe.

7. The apparatus of claim 1, wherein retrieving the auxiliary information for the one or more vitality data sets comprises:
retrieving the auxiliary information using an auxiliary module, wherein the auxiliary module is configured to:
receive the one or more vitality data sets;
determine at least one missing element within the one or more vitality data sets; and
retrieve the auxiliary information as a function of the missing element.

8. The apparatus of claim 1, wherein retrieving the auxiliary information comprises retrieving the auxiliary information as a function of a web crawler.

9. The apparatus of claim 1, wherein identifying the at least one cluster based on the one or more augmented data sets further comprises classifying the one or more augmented data sets to the at least one cluster as a function of a classifier machine learning model.

10. The apparatus of claim 1, wherein providing the at least one cluster to a cluster analysis platform comprises:
generating prediction training data comprising a plurality of augmented data sets correlated to a plurality of predictive outputs;
iteratively training a prediction machine learning model as a function of the prediction training data and the similarity datum; and
determining the predictive outputs using the prediction machine learning model.

11. A method for identifying clusters based on augmented data sets, the method comprising:
receiving, by at least a processor, one or more vitality data sets;
retrieving, by at least a processor, auxiliary information for the one or more vitality data sets;
generating, by at least a processor, one or more augmented data sets as a function of the auxiliary information and the one or more vitality data sets;
identifying, by at least a processor, at least one cluster based on the one or more augmented data sets; and
providing, by at least a processor, the at least one cluster to a cluster analysis platform, wherein the cluster analysis platform is configured to generate a similarity datum as a function of the at least one cluster and the one or more augmented data sets.

12. The method of claim 11, wherein:
the one or more vitality data sets comprise at least a data file; and
generating the one or more augmented data sets comprises identifying textual data from the at least a data file using optical character recognition.

13. The method of claim 11, wherein the cluster analysis platform comprises a graphical user interface configured to present the similarity datum in a graphical format.

14. The method of claim 11, wherein the auxiliary information comprises one or more quantitative data points used to quantify one or more elements within the one or more vitality data sets.

15. The method of claim 14, wherein retrieving the auxiliary information comprises:
identifying one or more treatment events with each of the one or more vitality data sets; and
retrieving the one or more quantitative data points associated with each of the one or more vitality data sets for each treatment event of the one or more treatment events.

16. The method of claim 15, wherein:
identifying the one or more treatment events comprises generating a timeframe for each treatment event of the one or more treatment events; and
retrieving the one or more quantitative data points associated with each of the one or more vitality data sets for each treatment event comprises grouping the one or more quantitative data points for each treatment event as a function for the timeframe.

17. The method of claim 11, wherein retrieving the auxiliary information for the one or more vitality data sets comprises:
retrieving the auxiliary information using an auxiliary module, wherein the auxiliary module is configured to:
receive the one or more vitality data sets;
determine at least one missing element within the one or more vitality data sets; and
retrieve the auxiliary information as a function of the missing element.

18. The method of claim 11, wherein retrieving the auxiliary information comprises retrieving the auxiliary information as a function of a web crawler.

19. The method of claim 11, wherein identifying the at least one cluster based on the one or more augmented data sets further comprises classifying the one or more augmented data sets to the at least one cluster as a function of a classification machine learning model.

20. The method of claim 11, wherein providing the at least one cluster to a cluster analysis platform comprises:
generating prediction training data comprising a plurality of augmented data sets correlated to a plurality of predictive outputs;
iteratively training a prediction machine learning model as a function of the prediction training data and the similarity datum; and
determining the predictive outputs as a function of the prediction machine learning model.
